(19) United States
(12) Patent Application Publication
(10) Pub. No.: US 2023/0355733 A1
(43) Pub. Date: Nov. 9, 2023

(54) APC targeting units for immunotherapy

(71) Applicant: Evaxion Biotech A/S, Hørsholm (DK)

(72) Inventors: Birgitte Rønø, Hørsholm (DK); Marina Barrio Calvo, Hørsholm (DK); Anders Bundgård Sørensen, Hørsholm (DK); Christian Thygesen, Hørsholm (DK); Jens Kringelum, Hørsholm (DK); Stine Friis, Hørsholm (DK)

(21) Appl. No.: 18/004,889

(22) PCT Filed: Jul. 14, 2021
(86) PCT No.: PCT/EP2021/069582

§ 371 Date: Jan. 10, 2023

(30) Foreign Application Priority Data
Jul. 14, 2020 (EP) 20185743.0
Nov. 13, 2020 (EP) 20207526.3
Apr. 29, 2021 (EP) 21171319.3

(51) Int. Cl. A61K 39/00; A61K 39/215; A61P 37/04

(52) U.S. Cl. CPC A61K 39/001121; A61K 39/215; A61P 37/04; A61K 2039/627; A61K 2039/70

(57) ABSTRACT

Immune therapy and vaccines, such as cancer immunotherapy, or vaccines for infections with microorganisms, such as a bacterial or viral infection. In particular, the present invention relates to methods and products for prophylactic or treating cancer or infections with microorganisms by administration of specific fusion polypeptides or nucleic acids encoding such fusion polypeptides.

### FIELD OF THE INVENTION

The present invention relates to immune therapy, such as cancer immunotherapy, or immune therapy & vaccines for infections with microorganisms, such as a bacterial or viral infection.

In particular, the present invention relates to methods and products for prophylactic or treating cancer or infections with microorganisms by administration of specific fusion polypeptides or nucleic acids encoding such fusion polypeptides.

### BACKGROUND OF THE INVENTION

Treatment of malignant neoplasms in patients has traditionally focused on eradication/removal of the malignant tissue via surgery, radiotherapy, and/or chemotherapy using cytotoxic drugs in dosage regimens that aim at preferential killing of malignant cells compared to killing of non-malignant cells.

In addition to the use of cytotoxic drugs, more recent approaches have focused on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance proposed by Burnet and Thomas suggested that lymphocytes recognize and eliminate autologous cells—including cancer cells—that exhibit altered antigenic determinants, and it is today generally accepted that the immune system inhibits carcinogenesis to a high degree. Nevertheless, immunosurveillance is not 100% effective and it is a continuing task to device cancer therapies where the immune system's ability to eradicate cancer cells is sought improved/stimulated.

One approach has been to induce immunity against cancer-associated antigens, but even though this approach has the potential of being promising, it suffers the same drawback as antibody therapy that only a limited number of antigens can be addressed.

Many if not all tumours express mutations. These mutations potentially create new targetable antigens (neo-antigens), which are potentially useful in specific T cell immunotherapy if it is possible to identify the neo-antigens and their antigenic determinants within a clinically relevant time frame. Since it with current technology is possible to fully sequence the genome of cells and to analyse for existence of altered or new expression products, it is possible to design personalized vaccines based on neo-antigens. However, attempts at providing satisfactory clinical results have as of today largely failed.

One mode of vaccination that has been investigated in detail since the early 1990'ies is nucleic acid vaccination (also termed DNA vaccination), where DNA is administered in a non-viral plasmid form to somatic cells of a mammal leading to expression of the genes comprised in the plasmid; in DNA vaccination the encoded material is immunogenic polypeptide(s), which upon production by the somatic cells will be able to induce an immune response. This approach is appealing as it avoids the need of producing the protein immunogen in clinical grade purity using expensive recombinant expression systems. However, it has proven difficult to obtain expression levels from the DNA administered which are high enough to effect satisfactory immune responses in humans.

Antigen-presenting cells (APC) are vital for effective adaptive immune response and are cells that display antigen complexed with major histocompatibility complexes (MHCs) on their surfaces. The cells include macrophages, B cells and dendritic cells, and present foreign antigens to helper T cells. Also virus-infected cells or cancer cells can present antigens originating inside the cell to cytotoxic T cells. Consequently, targeting antigen-presenting cell offers opportunities to induce superior immune responses.

There is an existing need for provision of improved or effective vaccines against foreign agents, such as microorganisms including bacteria and virus, as well as for vaccines against neoplastic cells, such as for anti-cancer vaccines, in particular nucleic acid vaccines that can effectively target bacterial and viral antigens as well as neo-antigens and induce clinically significant immune responses in vaccinated human beings.

### OBJECT OF THE INVENTION

It is an object of embodiments of the invention to provide polypeptides constructs and nucleic acid molecules encoding such polypeptide constructs with superior anti-microorganism or anti-tumor effect and which constructs are able to elicit even higher T cell responses than known vaccines.

It is a further object of embodiments of the invention to provide polypeptides constructs and nucleic acid molecules encoding such polypeptide constructs designed to direct and assist in APC uptake of antigenic units of bacterial or viral origin, or cancer neo-epitopes, optionally activation of the APC followed by cytokine cascade enabling the provision of a very effective protective immunity against the cancer, bacterial or virus.

It is a further object of embodiments of the invention to provide methods for the use of such polypeptides constructs and nucleic acid molecules encoding such polypeptide constructs.

### SUMMARY OF THE INVENTION

The present inventors have designed novel and improved polypeptides constructs and nucleic acid molecules encoding such polypeptide constructs generating a next generation nucleic acid (DNA or RNA) immunotherapy, such as neoepitope immunotherapy or immunotherapy targeting infectious agents, utilising an antigen presenting cell (APC)—targeting unit.

It has been found by the present inventor(s) that targeting of APC may be used to enhance the immunotherapeutic effects while maintaining antitumor activity of a neo-epitope vaccine, which enable a superior anti-tumour effect eliciting even higher T cell responses than known vaccines. It has also been found that viral antigens, such as the SARS-CoV-2 spike protein can be successfully addressed with the essentially same technology.

So, in a first aspect the present invention relates to fusion polypeptides comprising
i) at least one antigenic unit;
ii) at least one antigen presenting cell (APC) targeting unit, which is targeting dendritic cells;
iii) optionally a multimerization, such as a dimerization unit, which unit provides for the multimerization of said fusion polypeptide to comprise two or more antigenic units and two or more antigen presenting cell (APC) targeting units. In a second aspect the present invention relates to an expression vector, which comprises a sequence of nucleotides encoding a fusion polypeptide according to the present invention.

In a further aspect the present invention relates to a system of at least two expression constructs comprising i) a first expression construct comprising a sequence of nucleotides encoding at least one antigenic unit, and ii) a second expression construct comprising a sequence of nucleotides encoding at least one antigen presenting cell (APC) targeting unit, which is targeting dendritic cells.

In a further aspect the present invention relates to a method for the treatment of a disease characterized by exhibiting a specific disease antigenic unit, such as an epitope that are not exhibited by normal cells in the patient, or which epitope is foreign to the patient, the method comprising administering an immunogenically effective amount of a composition comprising a fusion polypeptide according to the present invention, or which composition is comprising at least one expression vector according to the present invention, or the system of at least two expression constructs according to the present invention, whereby somatic cells in the patient are brought to express the sequence of nucleotides contained within the expression vector; the method optionally further comprising administering a pharmaceutically acceptable carrier, diluent, or excipient.

In a further aspect the present invention relates to a method for the treatment of a neoplasm, such as a malignant neoplasm or for inducing a therapeutic or ameliorating immune response against such neoplasm, in a mammalian patient, wherein the neoplasm exhibits epitopes (neo-epitopes) that are not exhibited by non-neoplastic cells in the patient, the method comprising administering an immunogenically effective amount of a composition comprising a fusion polypeptide according to the present invention, or which composition is comprising at least one expression vector according to the present invention, or the system of at least two expression constructs according to the present invention, whereby somatic cells in the patient are brought to express the sequence of nucleotides contained within the expression vector; the method optionally further comprising administering a pharmaceutically acceptable carrier, diluent, or excipient.

In a further aspect the present invention relates to a method for the prophylactic or treatment of a bacterial or viral infection, which bacteria or virus is characterized by exhibiting a specific antigenic unit, and/or a set of epitopes, of said bacteria or virus, the method comprising administering an immunogenically effective amount of a composition comprising a fusion polypeptide according to the present invention, or which composition is comprising at least one expression vector according to the present invention, or the system of at least two expression constructs according to the present invention, whereby somatic cells in the patient are brought to express the sequence of nucleotides contained within the expression vector; the method optionally further comprising administering a pharmaceutically acceptable carrier, diluent, or excipient. Preferably said viral infection is infection with 3 coronavirus, such as SARS coronavirus, and in particular SARS-CoV-2—in such embodiments, the fusion polypeptide can be any one of those disclosed supra, where the antigen unit is derived from a β coronavirus.

In some embodiments the patient is a human being. In some embodiments the immunogenically effective amount of a composition is administered parenterally, such as via the intramuscular route, the intradermal route, transdermal route, the subcutaneous route, the intravenous route, the intra-arterial route, the intratechal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

In some embodiments the pharmaceutically acceptable carrier, diluent, or excipient is an aqueous buffered solution. In some embodiments the aqueous buffered solution is Tyrode's buffer. In some embodiments the Tyrode's buffer has the composition 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose. In some embodiments the concentration of Tyrode's buffer is about 35% v/v. In some embodiments the aqueous buffer is phosphate-buffered saline (PBS) buffer.

In some embodiments the method comprises administering an immunogenically effective amount of a composition comprising at least one expression vector as defined in any one of claims 10-15 with an effective dosage between 0.1 μg and 25 mg of the expression vector, such as between 0.5 μg and 20 mg, between 5 μg and 15 mg, between 50 μg and 10 mg, and between 500 μg and 8 mg, in particular about 0.0001, about 0.0005, about 0.001, about 0.005, about 0.01, about 0.05, about 0.1, about 0.5, about 1, about 2, about 3, about 4, about 5, about 6, about 7 and about 8 mg.

In some embodiments the method comprises administering an immunogenically effective amount of a composition which composition further comprises an effective amount of an amphiphilic block co-polymers comprising blocks of poly(ethylene oxide) and polypropylene oxide), such as Kolliphor® P188.

### LEGENDS TO THE FIGURE

FIG. 1: Illustration of one suggested design of a suitable fusion polypeptide and DNA encoding it. In this example the construct contains an APC targeting unit being CCL3 or other cytokine/chemokine; a dimerization unit containing Hinge (h1 and h4) and CH3 from IgG3; and antigenic units being neo-epitopes.

FIG. 2: Illustration of mechanism of action for the DNA vaccine as illustrated in FIG. 1.

FIG. 3: Illustration of different APC targeting units for fusion polypeptide designs according to the invention. A) illustrates the constructs of example 1, whereas B) illustrates the constructs of example 2.

FIG. 4: Plasmid map of pUMVC4 from Aldevron.

Details are provided in the examples and SEQ ID NO: 29.

FIG. 5: Plasmid map of pUMVC4 mCCL19 S16A as example of an APC targeting design. The pUMVC4 vector is containing an insert encoding a kozak sequence, murine CCL19 as APC targeting unit, hinge 1, hinge 4 and Ch3 from human IgG followed by the 5 neoepitopes C22, C23, C38, C25, C30.

FIG. 6: Tumour volume reduction in mice vaccinated with Kolliphor® and vaccine plasmids. APC targeting vaccines as compared to control groups (untreated mice or mice treated with empty mock plasmid). *: p<0.05 (Kruskal-Wallis test). See example 1 for details.

FIG. 7: Diagram shows detection of C22 MHC I multimers.

The graph shows the frequency of murine CD8+ T cells reactive with the C22 peptide upon vaccination of mice with experimental DNA vaccines. See example 1 for details.

FIG. 8: IFN-γ production in T cells from mice vaccinated with Kolliphor® and vaccine plasmids. See example 1 for details.

FIG. 9: Tumour volume reduction in mice vaccinated with Kolliphor® and vaccine plasmids. See example 2 for details.

FIG. 10: Diagram shows detection of C22 MHC I multimers.

The graph shows the frequency of murine CD8+ T cells reactive with the C22 peptide upon vaccination of mice with experimental DNA vaccines. See example 2 for details.

FIG. 11: IFN-γ production in T cells from mice vaccinated with Kolliphor® and vaccine plasmids. See example 2 for details.

FIG. 12: Tumour volume reduction in mice vaccinated with Kolliphor® and vaccine plasmids. See example 3 for details.

FIG. 13: Diagram shows detection of C22 MHC I multimers. The graph shows the frequency of murine CD8+ T cells reactive with the C22 peptide-loaded MHCI tetramer upon vaccination of mice with experimental DNA vaccines. See example 3 for details.

FIG. 14: IFN-γ production in T cells from mice vaccinated with Kolliphor® and vaccine plasmids. See example 3 for details.

FIG. 15: Illustration of DNA designs encoding the separate units of the fusion protein according to the invention. Illustrates the constructs of example 4.

FIG. 16: Tumour volume reduction in mice vaccinated with Kolliphor® and vaccine plasmids. See example 4 for details.

FIG. 17: Diagram shows detection of C22 MHC I multimers. The graph shows the frequency of murine CD8+ T cells reactive with the C22 peptide-loaded MHCI tetramer upon vaccination of mice with experimental DNA vaccines. See example 4 for details.

FIG. 18: DNA designs according to the invention.

FIG. 19: Schematic representation of designs for selected APC targeting candidates.

FIG. 20: Schematic representation of fusion proteins of the present invention.

FIG. 21: Plasmid map of vector of the invention.

FIG. 22: Bar graph showing IFN-γ release from splenocytes in ELISPOT assay.

FIG. 23: Scatter plots and bar graph showing titers of antibodies induced by a construct of the invention.

FIG. 24: Bar graph showing comparison between SARS-CoV-2 neutralizing activities of sera.

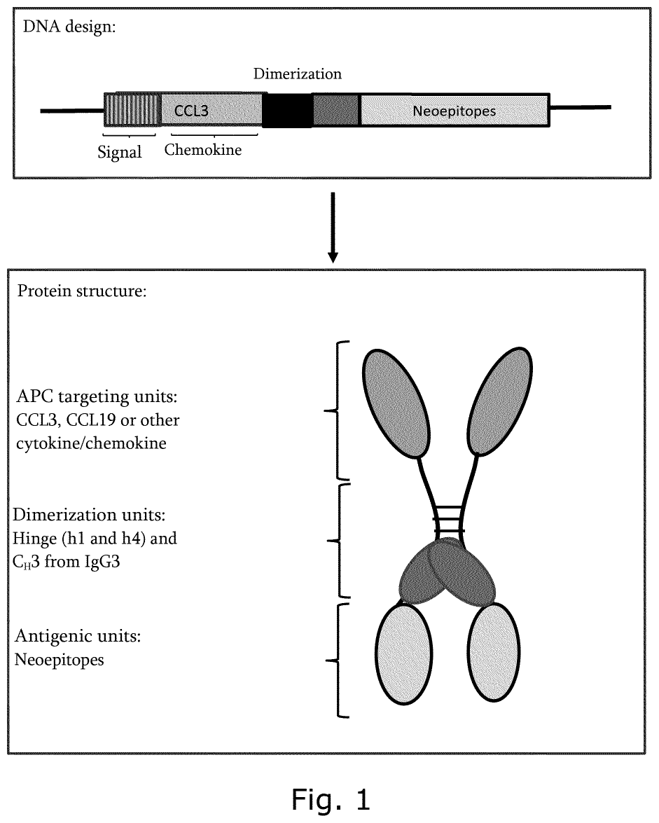

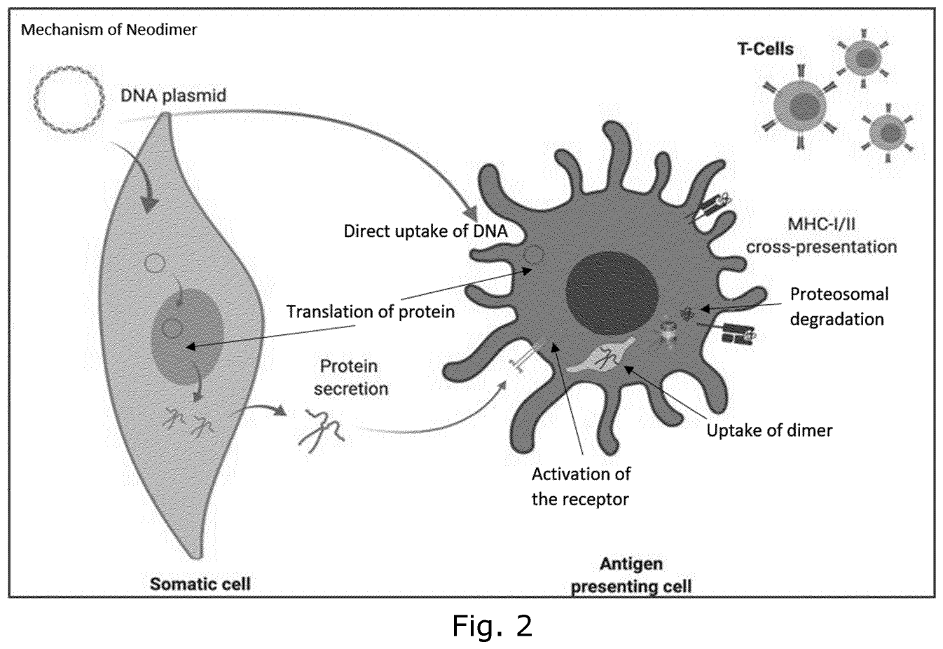

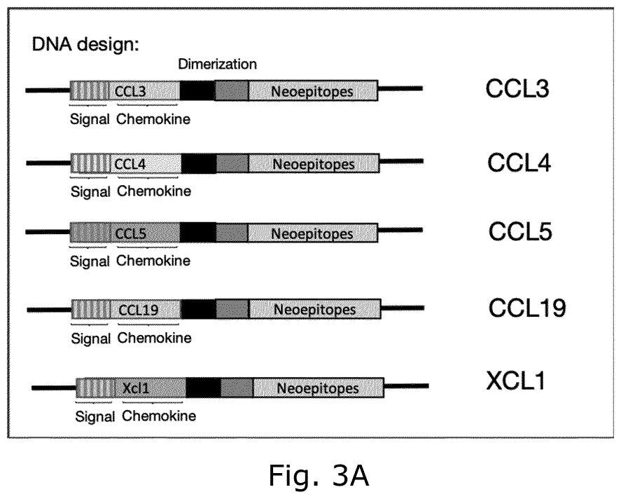

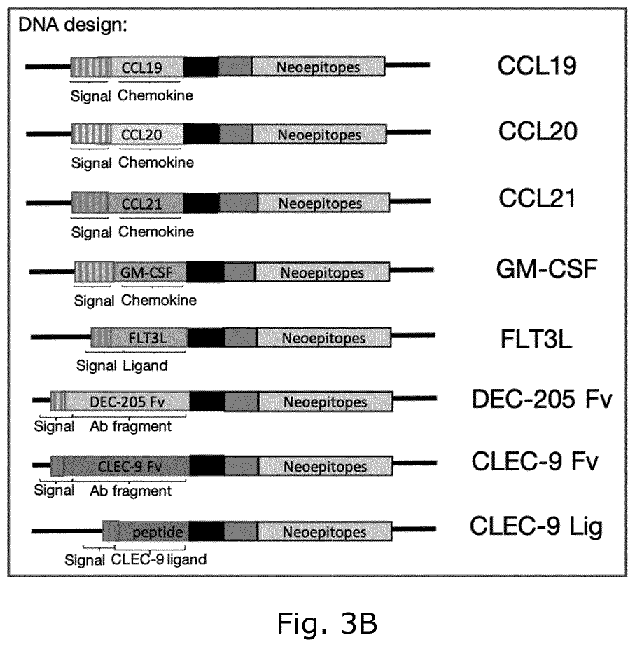

### DETAILED DISCLOSURE OF THE INVENTION

A DNA immunotherapy or vaccine, such as a DNA neo-epitope immunotherapy containing an APC targeting element targeting dendritic cells is expected to have superior effect, such as an anti-tumor effect and may elicit higher T cell responses, than a DNA technology without, due to either i) directing and assisting in APC uptake of the neo-epitopes, and/or ii) activation of the APC followed by cytokine cascade.

The fusion polypeptide constructs according to the present invention comprise an antigenic unit, such as cancer, bacterial or viral antigen antigenic units, such as bacterial or viral antigen antigenic units comprising epitopes of this microorganism, or an antigenic unit comprising epitopes, neo-epitopes, such as T cell epitopes that are not exhibited by non-neoplastic cells in the patient.

Antigenic units may be derived from virus or from bacteria. Examples of viruses from where antigenic units may derived and for use with the fusion polypeptides of the present invention include, but are not limited to, e.g., HIV, HCV, CMV, HPV, Influenza, adenoviruses, retroviruses, picornaviruses, coronaviruses etc. Non-limiting example of retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NSI, NSI, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens.

The antigenic units to be incorporated into the fusion polypeptides according to the present invention may be derived from an adenovirus, retrovirus, picornavirus, herpesvirus, rotavirus, hantavirus, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus. In certain specific, non-limiting examples, the viral antigen are peptides obtained from at least one of HIV, CMV, hepatitis A, B, and C, influenza, measles, polio, smallpox, rubella; respiratory syncytial, herpes simplex, varicella zoster, Epstein-Barr, Japanese encephalitis, rabies, Influenza, and/or cold viruses.

The term “antigenic unit” denotes the region of a substance of matter, such as the part of a foreign organism which is recognized by the immune system's specifically recognizing components (antibodies, T-cells).

A “neo-epitope” is an antigenic determinant (typically an MHC Class I or II restricted epitope), which does not exist as an expression product from normal somatic cells in an individual due to the lack of a gene encoding the neo-epitope, but which exists as an expression product in mutated cells (such as cancer cells) in the same individual. As a consequence, a neo-epitope is from an immunological viewpoint truly non-self in spite of its autologous origin and it can therefore be characterized as a tumour specific antigen in the individual, where it constitutes an expression product. Being non-self, a neo-epitope has the potential of being able to elicit a specific adaptive immune response in the individual, where the elicited immune response is specific for antigens and cells that harbour the neo-epitope. Neo-epitopes are on the other hand specific for an individual as the chances that the same neo-epitope will be an expression product in other individuals is minimal. Several features thus contrast a neo-epitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neo-peptide” is a peptide (i.e. a polyamino acid of up to about 50 amino acid residues), which includes within its sequence a neo-epitope as defined herein. A neo-peptide is typically “native”, i.e. the entire amino acid sequence of the neo-peptide constitutes a fragment of an expression product that can be isolated from the individual, but a neo-peptide can also be “artificial”, meaning that it is constituted by the sequence of a neo-epitope and 1 or 2 appended amino acid sequences of which at least one is not naturally associated with the neo-epitope. In the latter case the appended amino acid sequences may simply act as carriers of the neo-epitope, or may even improve the immunogenicity of the neo-epitope (e.g. by facilitating processing of the neo-peptide by antigen-presenting cells, improving biologic half-life of the neo-peptide, or modifying solubility).

A “neo-antigen” is any antigen, which comprises a neo-epitope. Typically, a neo-antigen will be constituted by a protein, but a neo-antigen can, depending on its length, also be identical to a neo-epitope or a neo-peptide.

The term “amino acid sequence” is the order in which amino acid residues, connected by peptide bonds, lie in the chain in peptides and proteins. Sequences are conventionally listed in the N to C terminal direction.

The fusion polypeptide constructs according to the present invention further comprise at least one antigen presenting cell (APC) targeting unit.

Antigen-presenting cells (APC) are cells that displays antigen complexed with major histocompatibility complexes (MHCs) on their cell surfaces, a process known as antigen presentation. Specialized antigen-presenting cells include macrophages, B cells and dendritic cells, which present foreign antigens to helper T cells, while virus-infected cells (or cancer cells) can present antigens originating inside the cell to cytotoxic T cells. An antigen presenting cell (APC) targeting unit is any molecule or ligand that is suitable for the specific targeting to these APC, such as by specifically targeting different surface molecules on APCs.

Suitable targeting units to be used according to the invention includes the following as well as the corresponding human sequence:

| Molecule | Function | SEQ ID |
|---|---|---|
| mCCL3 | APC targeting | SEQ ID NO: 36 |
| mCCL4 | APC targeting | SEQ ID NO: 37 |
| mCCL5 | APC targeting | SEQ ID NO: 38 |
| mCCL19 | APC targeting | SEQ ID NO: 39 |
| mXcl1 | APC targeting | SEQ ID NO: 40 |
| mCCL20 | APC targeting | SEQ ID NO: 41 |
| mCCL21 | APC targeting | SEQ ID NO: 42 |
| GM-CSF | APC targeting | SEQ ID NO: 43 |
| Secretion signal, mouse | secretion signal | SEQ ID NO: 44 |
| serum albumin | | |
| anti-murine DEC-205 Fv | APC targeting | SEQ ID NO: 45 |
| anti-murine CLEC9 Fv | APC targeting | SEQ ID NO: 46 |
| CLEC9 ligand | APC targeting | SEQ ID NO: 47 |
| mFLT3L | APC targeting | SEQ ID NO: 48 |

Suitable targeting units to be used according to the invention is disclosed in any one of Takashi Sato et al. Blood. 2011 Mar. 24; 117(12): 3286-3293; Cagan Gurer et al. Blood. 2008 Aug. 15; 112(4): 1231-1239; Wan-Lun Yan et al. Immunotherapy (2017) 9(4), 347-360; Gerty Schreibelt et al. BLOOD, 8 Mar. 2012, VOLUME 119, NUMBER 10; and Zhongyi Yan et al. Oncotarget, Vol. 7, No. 26, May 2016, p. 40437.

### Definitions

As used herein “immature dendritic cells (imDCs)” refers to a subtype of primitive immature dendritic cells characterized by expression of one or more classical dendritic cell surface markers CD1a, CD11c, CD11b, CD40, HLADR, and major histocompatibility complex class II (MHC-II).

As used herein “mature dendritic cells (mDCs)” refers to a subtype of dendritic cells characterized by upregulated levels of surface maturation markers such as CD80, CD83, and CD86.

An “expression construct” is in the present context a nucleotide sequence, which comprises the necessary genetic elements, which allows a host cell to express a coding sequence present in the expression construct—as such, the expression construct is integrated into an expression vector, typically a plasmid or virus. An expression construct will as a minimum include a promoter/enhancer regions and a coding sequence initiated by a start codon and terminated by a stop codon. In addition the expression construct may comprise a ribosomal binding site, and a transcription termination sequence. In addition, various regulatory elements can be included in the expression construct.

As used herein a “linker” refers to any compound suitable for assembly of the two or more different or identical linear peptide sequences or subunits into a multimeric polypeptide. The term includes any linker found useful in peptide chemistry. Since the multimeric polypeptide or fusion polypeptide may be assembled or connected by standard peptide bonds in a linear way, the term linker also includes a “peptide spacer”, also referred to as a “spacer”. It is to be understood that linkers may be used both to separate encoded neo-epitopes in the fusion polypeptides of the invention, or linkers may be used to separate the neo-epitope units of the fusion polypeptide from the antigen presenting cell (APC) targeting unit of the fusion polypeptide.

A linker may be “rigid”, meaning that it does substantially not allow the two amino acid sequences that it connects to move freely relative to each other. Likewise, a “flexible” linker allows the two sequences connected via the linker to move substantially freely relative to each other. In encoded expression products that contain more than one neo-epitope, both types of linkers are useful.

Linkers of interest, which can be encoded by an expression vector used in the invention, are listed in the following table:

| Type | Name | Sequence |
|---|---|---|
| Flexible | FS | GSGGGA (SEQ ID NO: 1) |
| Flexible | FL | GSGGGAGSGGGA (SEQ ID NO: 2) |
| Flexible | FV1 | GSGGGAGSGGGAGSGGGA (SEQ ID NO: 3) |
| Flexible | FV2 | GSGGGAGSGGGAGSGGGAGSGGGA (SEQ ID |
| | | NO: 4) |
| Flexible | FM | GENLYFQSGG (SEQ ID NO: 5) |
| Rigid | RL1 | KPEPKPAPAPKP (SEQ ID NO: 6) |
| Rigid | RL2 | AEAAAKEAAAKA (SEQ ID NO: 7) |
| Rigid | RM | SACYCELS (SEQ ID NO: 8) |
| Flexible | | SGGGSSGGGS (SEQ ID NO: 9) |
| Flexible | | GGGGSGGGGS (SEQ ID NO: 10) |
| Flexible | | SSGGGSSGGG (SEQ ID NO: 11) |
| Flexible | | GGSGGGGSGG (SEQ ID NO: 12) |
| Flexible | | GSGSGSGSGS (SEQ ID NO: 13) |
| Hinge | H1 | ELKTPLGDTTHT (SEQ ID NO: 59) |
| Hinge | H4 | EPKSCDTPPPCPRCP (SEQ ID NO: 60) |
| Flexible | | GGGSSGGGSG (SEQ ID NO: 70) |
| Flexible | | GGGSS (SEQ ID NO: 71) |
| Flexible | | GGGSSGGGSSGGGSS (SEQ ID NO: 72) |
| Flexible | | GLGGL (SEQ ID NO: 73) |
| Flexible | | GLGGLAAA (SEQ ID NO: 74) |

In some embodiments, the linker is a peptide sequence. In some embodiments, the linker is not a peptide sequence. In some embodiments, the linker is not a branched peptide sequence.

In some embodiments, the linker does not itself contain a peptide sequence derived from or identical to the neo-epitope sequence and/or the antigen presenting cell (APC) targeting unit.

In some embodiments, the linker is derived from an immunoglobulin molecule (Ig), such as from IgG.

In some embodiments, the linker is or comprises a hinge region, such as a flexible hinge region, such as a hinge region derived from an immunoglobulin molecule (Ig), such as from IgG.

Alternative suitable linkers to be used according to the invention:

| Molecule | Function | SEQ ID NO: |
|---|---|---|
| dHLX protein | dimerization | SEQ ID NO: 49 |
| hMHD2 (human IgM) | dimerization | SEQ ID NO: 51 |
| Collagen trimerization domain | Trimerization | SEQ ID NO: 52 |
| p53 synthetic protein | Tetramerization | SEQ ID NO: 53 |
| T4 fibritin domain | Trimerization | SEQ ID NO: 69 |

Accordingly, in some embodiments the linker comprises or consists of a hinge region derived from IgM, and/or comprises or consists of a dimerization motif derived from a sequence encoded by SEQ ID NO: 51.

In some embodiments the linker comprises or consists of a trimerisation domain, such as a Collagen trimerisation domain, such as a trimerisation domain derived from a sequence encoded by SEQ ID NO: 52.

In some embodiments the linker comprises or consists of a dimerization motif derived from hMHD2 or dHXL, optionally further comprising a hinge region such as H1 described herein.

In some embodiments the linker comprises or consists of a tetramerization domain, such as a domain derived from p53, such as a tetraimerization domain derived from a sequence encoded by SEQ ID NO: 53, optionally further comprising a hinge region such as H1 described herein.

Suitable linkers to be used according to the invention is also described in any one of: Ana Alvarez-Cienfuegos et al, Scientific Reports 2016, 6:28643 | DOI: 10.1038/srep28643; Victor J. Sanchez-Arevalo Lobo et al. Int. J. Cancer: 119, 455-462 (2006); Oliver Seifert et al. Protein Engineering, Design & Selection vol. 25 no. 10 pp. 603-612, 2012; and Jörg Willuda et al. THE JOURNAL OF BIOLOGICAL CHEMISTRY Vol. 276, No. 17, Issue of April 27, pp. 14385-14392, 2001.

“An immunogenic carrier” or “pharmaceutically acceptable carrier” as used herein is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “fusion polypeptide” is in the present context intended to mean a polypeptide containing polypeptide elements or amino acid sequences having intended different functions. The different polypeptide elements or amino acid sequences are connected through a linker, which may be just another amino acid sequence, or the different polypeptide elements or amino acid sequences of the fusion polypeptide may just be connected by standard peptide bonds in a linear way.

The expression vector used according to the present invention is typically and preferably comprised in or constituted by a plasmid, but other expression vectors can be employed. The composition of the present invention aims at ensuring delivery of “naked” DNA to cells, i.e. a DNA expression vector, which is not part of a bacterium or virus that would be able to effect introduction of the expression vector into the target cells. A vector useful in the present compositions and methods can thus be circular or linear, single-stranded or double stranded and can in addition to a plasmid also be e.g. a cosmid, mini-chromosome or episome.

Each coding (and expressible) region can be present on the same or on separate vectors; however, it is to be understood that one or more coding regions can be present on a single vector, and these coding regions can be under the control of a single or multiple promoters. This means that the expression vector can encode a separate peptide expression product for each encoded fusion polypeptide or that the expression vector can encode a plurality of peptide expression products, where at least some exhibit(s) several encoded fusion polypeptide, of which at least some optionally are separated by peptide linkers.

In other words, in some cases only one single expression vector is administered and expressed, and this expression vector may encode a plurality of separate proteinaceous expression products or as few as 2 or even one single expression product—in this context it is only relevant whether the encoded fusion polypeptide are satisfactorily presented to the immune system and the choice of their presence in separate on combined expression products is therefore of minor relevance. In preferred embodiments, the expression vector expresses at least or about 5, such as at least or about 10, at least or about 15, at least or about 20, at least or about 25, at least or about 30 proteinaceous expression products. Higher numbers are contemplated and the limit is primarily set by the number of neo-epitopes of the fusion polypeptides it is possible to identify from a particular neoplasm. It goes without saying that the number of encoded neo-epitopes of the fusion polypeptide in the expression vector(s) cannot exceed the number of neo-epitopes found in the relevant malignant tissue.

The use of peptide linkers to separate encoded neo-epitope expression products provides spatial separation between epitopes in the expression product of the fusion polypeptide. This can entail several advantages: linkers can ensure that each neo-epitope is presented in an optimized configuration to the immune system, but use of appropriate linkers can also minimize the problem that irrelevant immune responses are directed against “junctional epitopes” which emerge in the regions constituted by the C-terminal end of one neo-epitope and the adjacent N-terminal end of the next neo-epitope in a multi-epitope containing expression product.

Encoded peptide linkers can be either “flexible” or “rigid”, cf. the definition above, where preferred encoded linkers are set forth. Also, it is envisaged that the linker(s) used in the invention in some embodiments can be cleavable, that is, include (a) recognition site(s) for endopeptidase(s), e.g. endopeptidases such as furin, caspases, cathepsins etc.

The neo-epitopes encoded by the expression vector can be identified in a manner known per se: “deep sequencing” of the genome of the malignant cells and of the genome of healthy cells in the same individual or a standard healthy genome can identify expressed DNA sections that provide for potentially immunogenic expression products unique to the malignant cells. The identified DNA sequences can thereafter be codon-optimized (typically for expression by human cells) and included in the expression vector—either as separate expression regions of as part of larger chimeric constructs.

In order to optimize the identification and selection of the bacterial and viral epitopes or cancer neo-epitopes that are to be expressed by the vector, any of the prediction methods available for this purpose are in practice useful. One example of a state of the art prediction algorithm is NetMHCpan-4.0 (www.cbs.dtu.dk/services/NetMHCpan-4.0/; Jurtz V et al., J Immunol (2017), ji1700893; DOI: 10.4049/jimmunol.1700893). This method is trained on a combination of classical MS derived ligands and pMHC affinity data. Another example is NetMHCstabpan-1.0 (www.cbs.dtu.dk/services/NetMHCstabpan-1.0/; Rasmussen M et al., Accepted for J of Immunol, June 2016). This method is trained on a dataset of in vitro pMHC stability measurement using an assay where each peptide is synthesized and complexed to the MHC molecule in vitro. No cell processing is involved in this assay and the environment where the pMHC stability is measured is somewhat artificial. The method is in general less accurate than NetMHCpan-4.0. U.S. Pat. No. 10,055,540 describes a method for identification of neo-epitopes using classical MS detected ligands. Other patent application publications using similar technology are WO 2019/104203, WO 2019/075112, WO 2018/195357 (MHC Class II specific), and WO 2017 106638. Finally, MHCflurry: (DOI: doi.org/10.1016/j.cels.2018.05.014; https://github.com/openvax/mhcflurry) is like NetMHCpan trained on MS detected ligand data and pMHC affinities. A peptide-MHC Class II interaction prediction method is also disclosed in a recent publication Garde C et al., Immunogenetics, DOI: doi.org/10.1007/s00251-019-01122-z. In this publication, naturally processed peptides eluted from MHC Class II are used as part of the training set and assigned the binding target value of 1 if verified as ligands and 0 if negative.

Generally, these prediction systems employ artificial neural networks (ANNs): ANNs can identify non-linear correlations: Quantification of non-linear correlations is not an easy task, since it is difficult to calculate by simple calculation. This is primarily due to non-linear correlations described with more parameters than linear correlations and probably first appear when all features are considered collectively. Hence it is needed to take all features into account in order to catch the dependency across features.

In order to further improve the likelihood that the selected encoded neo-epitopes provide for an effective immune response, use can preferably be made of the technology disclosed in European patent application nos: 19197295.9 and 19197306.4, both filed on 13 Sep. 2019. These applications disclose technology, which enables that stability of binding between peptides and MHC molecules can be determined and which enables that stability of MHC binding of neo-epitopes is determined as part of the neo-epitope detection and selection. In brief, the data obtained from stability determinations are e.g. used as part of the training set for an ANN, and the ANN can subsequently rank identified peptides according to their predicted binding stabilities towards relevant MHC molecules.

When a nucleic acid vaccine is administered to a patient, the corresponding gene product (such as a desired antigen) is produced in the patient's body. In some embodiments, nucleic acid vaccine vectors that include optimized recombinant polynucleotides can be delivered to a human to induce a therapeutic or prophylactic immune response.

Plasmid and other DNA vectors are typically more efficient for gene transfer to muscle tissue. The potential to deliver DNA vectors to mucosal surfaces by oral administration has also been reported and DNA plasmids have been utilized for direct introduction of genes into other tissues than muscle. DNA vaccines have been introduced into animals primarily by intramuscular injection, by gene gun delivery, by jet injection (using a device such as a Stratis® device from PharmaJet), or by electroporation; each of these modes of administration apply to the presently disclosed method. After being introduced, the plasmids are generally maintained episomally without replication. Expression of the encoded proteins has been shown to persist for extended time periods, providing stimulation of both B and T cells.

In determining the effective amount of the vector to be administered in the treatment method disclosed herein, the physician evaluates vector toxicities, progression of the cancer to be treated, and the production of anti-vector antibodies, if any. Administration can be accomplished via single or divided doses and typically as a series of time separated administrations. In the methods disclosed herein, the effective human dose per immunization in a time-separated series is between 0.1 μg and 500 mg, with dosages between 0.1 μg and 25 mg of the expression vector being preferred. That is, in the practice of the method disclosed herein dosages of between 0.5 μg and 20 mg in humans are typically used, and dosages are normally between 5 μg and 15 mg, between 50 μg and 10 mg, and between 500 μg and 8 mg, and particular interesting dosages are of about 0.0001, about 0.0005, about 0.001, about 0.005, about 0.01, about 0.05, about 0.1, about 0.5, about 1, about 2, about 3, about 4, about 5, about 6, about 7 and about 8 mg.

A series of immunizations with effective dosages will typically constitute a series of 2, 3, 4, 5, 6, or more dosages. Multiple (e.g. >6) dosages may for instance be relevant in order to keep a malignant neoplasm in check for a prolonged period and in such a situation the exact choice of encoded neo-epitopes in the vaccine vector can be changed over time in response to changes in the genome and proteome of the malignant cells. When and if new neo-epitopes are produced by the malignant cells these can conveniently be included as targets for the vaccine.

The vaccine used in the method disclosed herein comprises one or more expression vectors; for instance, the vaccine may comprise a plurality of expression vectors each capable of autonomous expression of a nucleotide coding region in a mammalian cell to produce at least one immunogenic polypeptide. An expression vector often includes a eukaryotic promoter sequence, such as the nucleotide sequence of a strong eukaryotic promoter, operably linked to one or more coding regions. The compositions and methods herein may involve the use of any particular eukaryotic promoter, and a wide variety are known; such as a CMV or RSV promoter. The promoter can be heterologous with respect to the host cell. The promoter used may be a constitutive promoter. The promoter used may include an enhancer region and an intron region to improve expression levels, such as is the case when using a CMV promoter.

Numerous plasmids known in the art may be used for the production of nucleic acid vaccines. Suitable embodiments of the nucleic acid vaccine employ constructs using the plasmids VR1012 (Vical Inc., San Diego Calif.), pCMVI.UBF3/2 (S. Johnston, University of Texas), pTVG4 (Johnson et al., 2006, Vaccine 24(3); 293-303), pVAX1 (Thermo Fisher Scientific, see above and the Examples below), or pcDNA3.1 (InVitrogen Corporation, Carlsbad, Calif.) as the vector.

In addition, the vector construct can according to the present invention advantageously contain immunostimulatory sequences (ISS). The aim of using such sequences in the vaccine vector is to enhance T-cell response towards encoded neo-epitopes, in particular Th1 cell responses, which are elicited by adjuvants that incorporate agonists of the toll-like receptors TLR3, TLR7-TLR8, and TLR9, and/or cytosolic RNA receptors such as, but not limited to, RIG-1, MDA5 and LGP2 (Desmet et al. 2012. Nat. Rev. Imm. 12(7), 479-491)

One possibility of employing ISS is to mimic a bacterial infection activating TLR9 by stimulating with unmethylated CG-rich motifs (so-called CpG motifs) of six bases with the general sequence NNCGNN (which have a 20-fold higher frequency in bacterial DNA than in mammalian DNA) either as directly administered small synthetic DNA oligos (ODNs), which contain partially or completely phosphorothioated backbones, or by incorporating the CpG motifs in the DNA vector backbone. Immunostimulatory CpGs can be part of the DNA backbone or be concentrated in an ISS where the CpG sequence(s) typically will be positioned between the stop codon in the neo-epitope coding sequence and the poly-A tail encoding sequence (i.e. the ISS is located between the stop codon and the polyadenylation signal). However, since CpG sequences exert an effect irrespectively of their position in a longer DNA molecule, their position could in principle be anywhere in the vaccine vector as long as the presence of the CpG motif does not interfere with the vector's ability to express the coding regions of the vaccine antigen.

If present in the vaccine as separate ODNs, where the ODNs function as immunological adjuvants, CpG motif containing oligonucleotides are typically to be co-administered/formulated together with the DNA vaccine by the selected delivery technology and will typically constitute hexamers or longer multimers of DNA comprising the sequence NNCGNN or the reverse complementary sequence. Useful ODNs for this purpose are e.g. commercially available from InivoGen, 5 Rue Jean Rodier, F-31400, Toulouse, France, which markets a range of Class A, B, and C ODNs. Examples are:

ODN1585
SEQ ID NO: 14
(5′-ggGGTCAACGTTGAgggggg-3′),
ODN2216
SEQ ID NO: 15
(5′-ggGGGACGATCGTCgggggg-3′),
ODN2336
SEQ ID NO: 16
(5′- gggGACGACGTCGTGgggggg -3′),
ODN1668
SEQ ID NO: 17
(5′-tccatgacgttcctgatgct-3′),
ODN1826
SEQ ID NO: 18
(5′-tccatgacgttcctgacgtt-3′),
ODN2006
SEQ ID NO: 19
(5′-tcgtcgttttgtcgttttgtcgtt-3′),
ODN2007
SEQ ID NO: 20
(5′- tcgtcgttgtcgttttgtcgtt -3′),
ODNBW006
SEQ ID NO: 21
(5′-tcgacgttcgtcgttcgtcgttc-3′),
ODN D-SL01
SEQ ID NO: 22
(5′-tcgcgacgttcgcccgacgttcggta-3′),
ODN2395
SEQ ID NO: 23
(5′-tcgtcgttttcggcgcgcgccg-3′),
ODN M362
SEQ ID NO: 24
(5′-tcgtcgtcgttcgaacgacgttgat-3′),
ODN D-SL03
SEQ ID NO: 25
(5′-tcgcgaacgttcgccgcgttcgaacgcgg-3′),

In these 12 ODNs, upper case nucleotides are phosphodiesters, lower case nucleotides are phosphorothioates, and underlining denotes palindromic sequences.

When CpG sequences are present in the plasmid backbone (which thereby become “self-adjuvating”), any number of possible NNGCNN or NNCGNN sequences can according to the invention be present, either as identical sequences or in the form of non-identical sequences of the CpG motif, or in the form of palindromic sequences that can form stem-loop structures. For instance, the following CpG motifs are of interest: AACGAC and GTCGTT, but also CTCGTT, and GCTGTT. An example of the use of such CpG encoding sequences is the following sequence excerpt from the commercially available pTVG4 vaccine vector backbone . . . agatctaacgacaaaacgacaaaacgacaaggcgccagatctggcgtttcgttttagtcgttttgtcgttagatct . . . (SEQ ID NO: 26), where the underlined nucleotides constitute the CpGs that are present in the pTVG4 plasmid vector sequence.

Another possibility is to mimic an RNA viral infection to activate TLR3 by adding a double stranded(ds) RNA either as synthetic RNA oligos such as Poly I:C (polyinosinic-polycytidylic acid), poly IC:U12 (uridine substituted poly I:C), or in the form of synthetic RNA oligonucleotides (ORNs); addition of these RNA molecules to the vaccine is, as for the ODN approach, a way of obtaining an adjuvant effect. Alternatively, the dsRNA can be encoded in the DNA vector backbone, which will be transcribed into RNA after vaccination—in this case the DNA vaccine hence encodes the immunological adjuvant. This approach can include DNA sequences that encode hairpin RNA with lengths of up to 100 base pairs, where the sequence is unspecific. Also the DNA can simultaneously include ODNs and encode ORNs of known sequences; the DNA can thus both be transcribed into a double stranded RNA capable of activating TLR3 and/or cytosolic RNA receptors such as RIG-1, MDA5, and LGP2 while comprising an ODN to activate TLR9. Examples of specific DNA sequences that include/encode immune stimulating CpG and dsRNA are for instance 5′-GGTGCATCGA TGCAGGGGGG-3′ (SEQ ID NO: 27) and 5′-GGTGCATCGA TGCAGGGGGG TATATATATA TTGAGGACAG GTTAAGCTCC CCCCAGCTTA ACCTGTCCTT CAATATATA TATA-3′ (SEQ ID NO: 28) (ref: Wu et al. 2011, Vaccine 29(44): 7624-30).

When ISS are present in the DNA vaccine vector, it is possible—and advantageous—to combine the approach of using CpG motifs to activate TLR9 with the presence of coding sequences for immune stimulating RNA to activate TLR3 and/or cytosolic RNA receptors such as RIG-1, MDA5, and LGP2; cf. Grossmann C et al. 2009, BMC. Immunology 10:43 and Desmet et al. 2012. Nat. Rev. Imm. 12(7), 479-491. Likewise, incorporation of ORNs and ODNs in the vaccine as separate adjuvants (alone or in combination) may be combined with the incorporation of ISS of both types in the DNA vaccine vector.

As is the case for the CpG motifs, the DNA encoding the immune stimulatory RNA ISS will preferably be present between the stop codon and the polyadenylation signal but can be present in any part of the vector as long as this does not impair the production of the intended polypeptide expression product.

In some specific embodiments, ISS is/are comprised in the vaccine compositions, and in particular embodiments this is achieved by incorporating an immunologically active and pharmaceutically acceptable amount of poly I:C and/or poly IC:U12. Poly I:C is constituted by a mismatched double-stranded RNA (dsRNA) with one strand being a polymer of inosinic acid and the other strand a polymer of cytidylic acid. Poly IC:U12 is a variant of poly I:C where uridine is introduced into the Poly I:C strand. These two substances will in that context function as immunological adjuvants, i.e. substances that themselves do not elicit a specific adaptive immune response, but which enhances the specific adaptive immune response against the vaccine antigen (or in the present case, the encoded antigen).

Poly I:C or poly IC:U12 (such as Ampligen®) will preferably be present in the composition so as to arrive at an administered dosage of between 0.1 and 20 mg per administration of the effective dosage of the expression vector; that is, the amount present in the composition is adjusted so as to arrive at such dosages per administration. Preferably the administered dosage of poly I:C or poly IC:U12 is between 0.2 and 15 mg per administration of the effective dosage of the expression vector, such as between 0.3 and 12, 0.4 and 10 and 0.5 and 8 mg, preferably about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9 and 4.0 mg. Particularly preferred are in the range between 0.5 and 2.0 mg per administration.

In some specific embodiments the vaccine composition may comprise a amphiphilic block co-polymers comprising blocks of poly(ethylene oxide) and polypropylene oxide), such as poloxamers, i.e. nonionic triblock copolymers composed of a central hydrophobic chain of polyoxypropylene (poly(propylene oxide)) flanked by two hydrophilic chains of polyoxyethylene (poly(ethylene oxide)). One particular preferred amphiphilic block co-polymers is poloxamer 188 (Kolliphor® P188 from BASF). The administered dosage of amphiphilic block co-polymers may be between 0.2% w/v and 20% w/v per administration of the effective dosage of the expression vector, such as between 0.2% w/v and 18% w/v, such as between 0.2% w/v and 16% w/v, such as between 0.2% w/v and 14% w/v, such as between 0.2% w/v and 12% w/v, such as between 0.2% w/v and 10% w/v, such as between 0.2% w/v and 8% w/v, such as between 0.2% w/v and 6% w/v, such as between 0.2% w/v and 4% w/v, such as between 0.4% w/v and 18% w/v, such as between 0.6% w/v and 18% w/v, such as between 0.8% w/v and 18% w/v, such as between 1% w/v and 18% w/v, such as between 2% w/v and 18% w/v, such as between 1% w/v and 5% w/v, such as between 2% w/v and 4% w/v. Particularly preferred are in the range between 0.5% w/v and 5% w/v per administration.

Accordingly, the vaccine composition according to the present invention, such as a DNA vaccine composition may comprises a pharmacologically acceptable amphiphilic block co-polymer comprising blocks of poly(ethylene oxide) and polypropylene oxide, which is described in detail in the following:

The amphiphilic block co-polymer is described more generally under the definition heading, but the preferred the amphiphilic block co-polymer is a poloxamer or a poloxamine. Poloxamers only vary slightly with respect to their properties, but preferred are poloxamer 407 and 188, in particular poloxamer 188.

When the amphiphilic block co-polymer is poloxamine, the preferred type is a sequential poloxamine of formula (PEO-PPO)4-ED, where PEO is poly(ethylene oxide), PPO is poly(propylene oxide) and ED is an ethylenediaminyl group. These molecules attain an X-like shape where the PEO-PPO groups protrude from the central ethylenediaminyl group. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

When used in the method disclosed herein, the concentration of the amphiphilic block co-polymer in the vaccine composition may preferably between 2 and 5% w/v, such as about 3% w/v.

A “PEO-PPO” or amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (“poloxamers”), PPO PEO PPO, (PEO PPO-)4ED (a “poloxamine”), and (PPO PEO-)4ED (a “reverse poloxamine”), where “ED” is a ethylenediaminyl group.

A “poloxamer” is a linear amphiphilic block copolymer constituted by one block of poly(ethylene oxide) (“PEO”) coupled to one block of poly(propylene oxide) (“PPO”) coupled to one block of PEO, i.e. a structure of the formula EOa-POb-EOa, where EO is ethylene oxide, PO is propylene oxide, a is an integer ranging between 2 and 130, and b is an integer ranging between 15 and 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of Mw 1800 (corresponding to b 31 PPO) and approximately 80% (w/w) of PEO (corresponding to a 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) are X-shaped block copolymers that bear four PEO-PPO arms connected to a central ethylenediamine via bonds between the free OH groups in the PEO-PPO-groups and the primary amine groups in ethylenediamine, and “reverse poloxamine are likewise X-shaped block copolymers that bear four PPO-PEO arms connected to a central ethylenediamine via bonds between the free OH groups in the PPO-PEO-groups and the primary amine groups in ethylenediamine.

The nucleic acid vaccine can also encode a fusion product containing one or more immunogenic polypeptides containing neo-epitopes. Plasmid DNA can also be delivered using attenuated bacteria as delivery system, a method that is suitable for DNA vaccines that are administered orally. Bacteria are transformed with an independently replicating plasmid, which becomes released into the host cell cytoplasm following the death of the attenuated bacterium in the host cell.

DNA vaccines, including the DNA encoding the desired antigen, can be introduced into a host cell in any suitable form including, the fragment alone, a linearized plasmid, a circular plasmid, a plasmid capable of replication, an episome, RNA, etc. Preferably, the gene is contained in a plasmid. In certain embodiments, the plasmid is an expression vector. Individual expression vectors capable of expressing the genetic material can be produced using standard recombinant techniques.

Routes of administration include, but are not limited to, intramuscular, intranasal, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. In other words, the route of administration can be selected from any one of parenteral routes, such as via the intramuscular route, the intradermal route, transdermal route, the subcutaneous route, the intravenous route, the intra-arterial route, the intrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

Typical routes of administration include intramuscular, intraperitoneal, intradermal and subcutaneous injection. Genetic constructs may be administered by means including, but not limited to, traditional syringes, needleless injection devices, “microprojectile bombardment gene guns”, or other physical methods such as electroporation (“EP”), “hydrodynamic method”, or ultrasound. DNA vaccines can be delivered by any method that can be used to deliver DNA as long as the DNA is expressed and the desired antigen is made in the cell.

In some embodiments, a DNA vaccine composition disclosed herein is delivered via or in combination with known transfection reagents such as cationic liposomes, fluorocarbon emulsion, cochleate, tubules, gold particles, biodegradable microspheres, or cationic polymers. Cochleate delivery vehicles are stable phospholipid calcium precipitants consisting of phosphatidyl serine, cholesterol, and calcium; this nontoxic and noninflammatory transfection reagent can be present in a digestive system. Biodegradable microspheres comprise polymers such as poly(lactide-co-glycolide), a polyester that can be used in producing microcapsules of DNA for transfection. Lipid-based microtubes often consist of a lipid of spirally wound two layers packed with their edges joined to each other. When a tubule is used, the nucleic acid can be arranged in the central hollow part thereof for delivery and controlled release into the body of an animal.

A DNA vaccine can also be delivered to mucosal surfaces via microspheres. Bioadhesive microspheres can be prepared using different techniques and can be tailored to adhere to any mucosal tissue including those found in eye, nasal cavity, urinary tract, colon and gastrointestinal tract, offering the possibilities of localized as well as systemic controlled release of vaccines. Application of bioadhesive microspheres to specific mucosal tissues can also be used for localized vaccine action. In some embodiments, an alternative approach for mucosal vaccine delivery is the direct administration to mucosal surfaces of a plasmid DNA expression vector which encodes the gene for a specific protein antigen.

The DNA plasmid vaccines disclosed are formulated according to the mode of administration to be used. Typically, the DNA plasmid vaccines are injectable compositions, they are sterile, and/or pyrogen free and/or particulate free. In some embodiments, an isotonic formulation is preferably used. Generally, additives for isotonicity can include sodium chloride, dextrose, mannitol, sorbitol and lactose. In some embodiments, isotonic solutions such as phosphate buffered saline are preferred; one preferred solution is Tyrode's buffer. In some embodiments, stabilizers include gelatine and albumin. In some embodiments, a stabilizing agent that allows the formulation to be stable at room or ambient temperature for extended periods of time, such as LGS or other poly-cations or poly-anions is added to the formulation.

The second constituent in the composition disclosed herein is the pharmaceutically acceptable carrier, diluent, or excipient, which is preferably in the form of a buffered solution. Parenteral vehicles include sodium chloride solution, Ringer's dextrose and sodium chloride, lactated Ringer's or fixed oils. Intravenous vehicles include fluid and nutrient replenishers, electrolyte replenishers such as those based on Ringer's dextrose, and the like. Preservatives and antimicrobials include antioxidants, chelating agents, inert gases and the like. Preferred preservatives include formalin, thimerosal, neomycin, polymyxin B and amphotericin B.

In preferred embodiments, the buffered solution is the one known as “Tyrode's buffer”, and in preferred embodiments the Tyrode's buffer has the composition 140 mM NaCl, 6 mM KCl, 3 mM CaCl2), 2 mM MgCl2, 10 mM 4-(2-hydroxyethyl)-1-piperazineethanesulfonic acid (Hepes) pH 7.4, and 10 mM glucose. The concentration of the Tyrode's buffer (or alternatives) is typically about 35% v/v, but depending on the water content of suspended plasmids, the concentration may vary considerably—since the buffer is physiologically acceptable, it can constitute any percentage of the aqueous phase of the composition.

Furthermore, in preferred embodiments, the buffered solutions is the PBS, and in preferred embodiments the PBS has composition of composition 0.28 mg Potassium dihydrogen phosphate, 1.12 mg Disodium hydrogen phosphate dihydrate and 9.0 Sodium chloride per 1 ml solution.

Additional carrier substances may be included and can contain proteins, sugars, etc. Such carriers may be aqueous or non-aqueous solutions, suspensions, and emulsions. Examples of non-aqueous carriers are propylene glycol, polyethylene glycol, vegetable oils such as olive oil, and injectable organic esters such as ethyl oleate. Aqueous carriers include water, alcoholic/aqueous solutions, emulsions or suspensions, including saline.

### SPECIFIC EMBODIMENTS OF THE INVENTION

As described above the present invention relates to fusion polypeptide comprising
i) at least one antigenic unit;
ii) at least one antigen presenting cell (APC) targeting unit, which is targeting dendritic cells;
iii) optionally a multimerization, such as a dimerization unit, which unit provides for the multimerization of said fusion polypeptide to comprise two or more antigenic units and two or more antigen presenting cell (APC) targeting units.

In some embodiments the APC targeting unit consist of or comprises a sequence of amino acids of at least one epitope being a neo-epitope of neoplastic cells from a patient.

In some embodiments the APC targeting unit does not comprise a neo-epitope of neoplastic cells from a patient.

In some embodiments the APC targeting unit is a bacterial or viral antigen, such as an antigenic unit comprising a sequence of amino acids of at least one bacterial or viral epitope.

In some embodiments APC targeting unit is targeting mature dendritic cells (mDCs).

In some embodiments the APC targeting unit is selected from CCL19 and CCL21, such as the human forms.

In some embodiments the APC targeting unit is targeting the receptor CCR7.

In some embodiments the APC targeting unit is selected from CCL3, CCL4, CCL5, CCL20, or XCL1, such as the human forms.

In some embodiments the APC targeting unit is targeting immature dendritic cells (imDCs).

In some embodiments the APC targeting unit is targeting a receptor selected from CCR1, CCR3, CCR5, CCR6, and XCR1.

In some embodiments the APC targeting unit consists of or comprises an antibody binding region with specificity for target surface molecules on antigen presenting cells, such as CLEC9A or DEC205, such as an anti-CLEC9A, anti-DEC205, or variants thereof, such as anti-CLEC9A Fv, anti-DEC205 Fv.

In some embodiments the APC targeting unit consists of or comprises a ligand, such as CLEC9 peptide ligand.

In some embodiments the APC targeting unit is selected from Xcl1, GM-CSF, anti-DEC-205 Fv, anti-CLEC9 Fv, and CLEC9 ligand.

In some embodiments the APC targeting unit is a cytokine, such as GM-CSF, such as an APC targeting unit that binds CD116.

In some embodiments the antigenic unit is connected to the targeting unit through a linker, such as GS linker, such as linker with the amino acid sequence GSGSGSGSGS (SEQ ID NO: 13), or a linker derived from an immunoglobulin molecule (Ig), such as IgG, such as a linker which contributes to the multimerization through the formation of an interchain covalent bond. In some embodiments the linker is or comprises a hinge region, such as an Ig, such as an IgG-derived hinge region and contributes to the multimerization through the formation of an interchain covalent bond, such as a disulfide bridge and/or contributes to the multimerization through the formation of non-covalent interactions (van der Waals interactions, hydrophobic interactions, and hydrophilic interactions, including polar interactions and ion bonding. In some embodiments the linker comprises a carboxyterminal C domain (CH3 domain), such as the carboxyterminal C domain of Ig (Cy3 domain), or a sequence that is substantially homologous to said C domain, such as the CH3 domain of IgG3. In some embodiments the hinge and CH3 domain are connected by a sequence of amino acids GGGSS (SEQ ID NO: 66), such as in triplicate sequence of the amino acids GGGSS (i.e. the sequence GGGSSGGGSSGGGSS; SEQ ID NO: 72). An alternative link between the hinge and CH3 domains can be via the amino acid sequence GGGSSGGGSG (SEQ ID NO: 70). In some embodiments the linker comprises a dimerization motif or any other multimerization domain, which participate in the multimerization through hydrophobic interactions, such as through a CH3 domain. In some embodiments the linker comprises a hinge region comprising h1+h4 or h4 derived from IgG, such as an IgG2 or IgG3.

In some embodiments the at least one antigenic unit consist of or comprises at least or about 5, such as at least or about 10, at least or about 15, at least or about 20, at least or about 25, and at least or about 30 epitopes.

In some embodiments the at least one antigenic unit comprises at least one epitope, which exhibits an MHC binding stability, which is above average, such as in the top quartile, among epitopes/antigens identified in the neoplastic cells or in the infectious bacteria or virus.

In some embodiments the at least one antigenic unit is a bacterial or viral antigen.

In such embodiments, the antigenic unit can comprise an amino acid sequence derived from a β-corona virus (in particular from a spike protein), such as a SARS corona virus (in particular from a spike protein), and preferably from SARS-CoV-2, and in particular from the SARS-CoV-2 spike protein. A particularly interesting (and herein exemplified) embodiment entails that the amino acid sequence is the Ribosome binding protein (RBD) form the SARS-CoV-2 spike protein. Hence, in an important embodiment the amino acid sequence consists of or comprises residues 319-541 of NCBI reference sequence no: YP_009724390 or an amino acid sequence having at least 80% sequence identity therewith, such as at least 85, 86, 87, 88, 89, 90, 91, 92, 93, 94, 95, 96, 97, 98, or 99% sequence identity therewith.

In some embodiments the fusion protein according to the present invention comprises a multimerization unit, such as a dimerization unit, that enables the formation of dimers, trimers, tetramers, pentamers, or multimers of higher order. Examples of suitable multimerization units are provided supra in discussion of linkers useful in the invention and include those having the amino acid sequences SEQ ID NOs.: SEQ ID NO: 49, 51, 52, 53, and 69.

The present invention further relates to a system of at least two expression constructs comprising i) a first expression construct comprising a sequence of nucleotides encoding at least one antigenic unit, and ii) a second expression construct comprising a sequence of nucleotides encoding at least one antigen presenting cell (APC) targeting unit, which is targeting dendritic cells.

In some embodiments the first expression construct comprising a sequence of nucleotides encoding at least one antigenic unit as defined according to the present invention.

In some embodiments the second expression construct comprises a sequence of nucleotides encoding at least one antigen presenting cell (APC) targeting unit, which is targeting dendritic cells as defined as defined according to the present invention.

In some embodiments the first expression construct comprises a sequence of nucleotides encoding at least one antigenic unit and further comprises a sequence of nucleotides encoding a multimerization unit, such as a dimerization unit, which unit provides for the multimerization of said at least one antigenic unit.

In some embodiments the at least two expression constructs are expressed on the same expression vector, such as under the control of two different promotors.

In some embodiments the at least two expression constructs are expressed by at least two different vectors.

### Example 1

### Assessment of Chemokines as APC Targeting Unit for Delivery of Neoepitopes

The objectives of the study were to test the ability of the invention, an APC targeting DNA vaccine, to induce neo-peptide specific T cells, reduce tumor growth and to monitor the impact of the vaccine on the well-being of vaccinated mice.

Plasmids for DNA vaccination were based on the commercially available pUMVC4™ vector available from Aldevron.

pUMVC4™ is according to the manufacturer's documentation a 4479 kb plasmid vector, which allows high-copy number replication in E. coli and high-level transient expression of encoded protein of interest in most mammalian cells. The vector (see FIG. 4) contains the following elements:
1) a human cytomegalovirus immediate-early (CMV) promoter for high-level expression in a wide range of mammalian cells,
2) a rabbit beta-globulin polyadenylation signal for efficient transcription termination and polyadenylation of mRNA,
3) a kanamycin resistance gene for selection in E. coli and
4) an immunostimulatory sequence (ISS) from ampicillin resistance gene, which makes it ideal for eliciting an immune response in vivo

The entire sequence of the pUMVC4™ plasmid is set forth in SEQ ID NO: 29.

Five expression vectors generated with the pUMVC4 as backbone and an APC targeting unit guiding the neoepitopes in S16A was constructed. The various chemokines used as APC targeting units are listed in the following table:

| APC targeting unit | SEQUENCE ID NO |
|---|---|
| mCCL3 | SEQ ID NO: 36 |
| mCCL4 | SEQ ID NO: 37 |
| mCCL5 | SEQ ID NO: 38 |
| mCCL19 | SEQ ID NO: 39 |
| mXCL1 | SEQ ID NO: 40 |

The five S16A neoepitopes were first identified by whole exome sequencing of the mouse colon cancer cell line CT26 and normal tissue samples from BALB/c mice and by selecting peptides found only in the cancer cells. In the experiment, the ability of the mice to generate immune responses against the identified neoepitopes was evaluated.

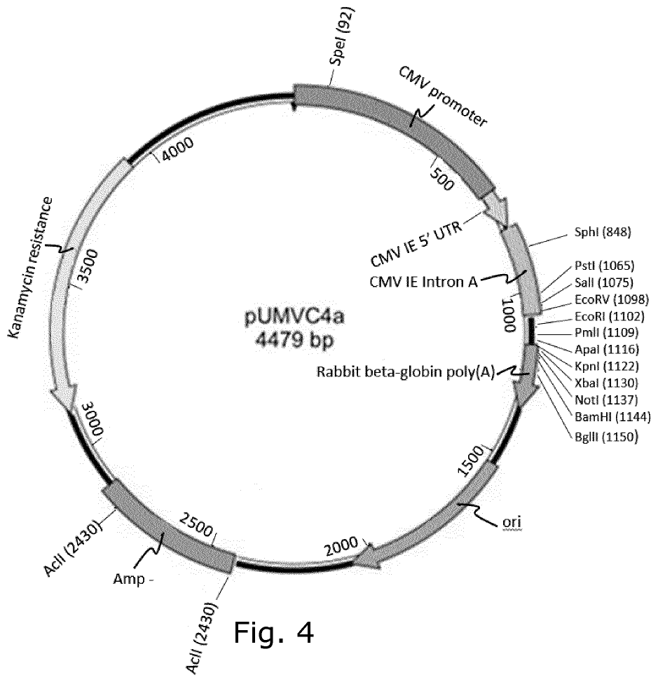

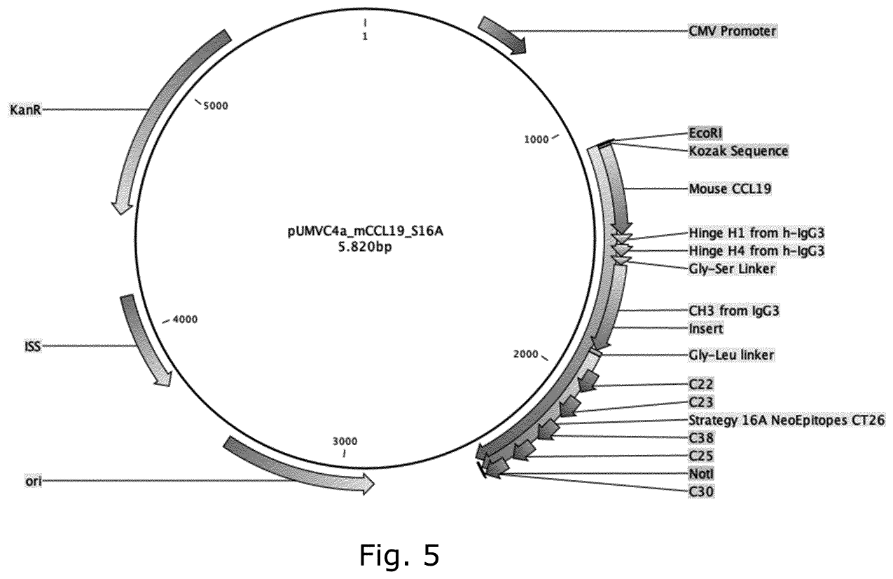

pUMVC4 APC-targeting S16A was constructed by ligating a DNA insert containing an APC targeting unit (SEQ IDs 36-43), human IgG3 hinge 1, hinge 4 and CH3 domain (SEQ ID NO: 31, 32 and 34) and codon-optimized (for expression in mice) DNA encoding a peptide containing the sequentially coupled 5 neo-epitopes C22, C23, C25, C30, and C38 (SEQ ID NOs: 61-65) into the pUMVC4 expression cassette, see FIG. 5. As control, a pUMVC4 plasmid containing an insert without an APC targeting unit, however encoding human IgG3 hinge 1, hinge 4 and CH3 domain and the same 5 neoepitopes C22, C23, C25, C30, and C38 was used (SEQ ID NOs: 61-65). In the pUMVC4 APC targeting S16A vectors, the inserts also included a Kozak consensus sequence to effectively initiate translation. The 5 neoepitope amino acid sequences used in the experiments (also in the following examples) are set forth in the following table:

| Peptide | AA Sequence | SEQ ID NO: |
|---|---|---|
| C22 | QIETQQRKFKASRASILSEMKMLKEKR | SEQ ID NO: 61 |
| C23 | VILPQAPSGPSYATYLQPAQAQMLTPP | SEQ ID NO: 62 |
| C25 | DTLSAMSNPRAMQVLLQIQQGLQTLAT | SEQ ID NO: 63 |
| C30 | DGQLELLAQGALDNALSSMGALHALPR | SEQ ID NO: 64 |
| C38 | RLHVVKLLASALSTNAAALTQELLVLD | SEQ ID NO: 65 |

Plasmids pUMVC4 mCCL3 S16A, pUMVC4 mCCL4 S16A, pUMVC4 mCCL5 S16A, pUMVC4 mCCL19 S16A, pUMVC4 mXcl1 S16A and (empty) pUMVC4 solubilised in sterile water were each mixed with poloxamer 188 (Kolliphor® from BASF) and Tyrode's buffer to obtain a composition of 3% w/v poloxamer 188 and 0.05 μg/μl plasmid in Tyrode's buffer.

The amphiphilic block co-polymers tested in combination with the DNA was Kolliphor® P188 (Or just referred to as Kolliphor in the present disclosure, or Lutrol® F 68), of the general formula:

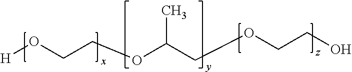

Study Plan

Mice received immunizations with the test vaccines on days −16, −9, −3, 5, and 12 relative to the CT26 tumour inoculation on day 0. Each immunization consisted of injection of 50 μl vaccine in the left and right tibia, respectively. Blood samples for C22 MHC I testing in a tetramer assay were obtained from the test animals on day 7 after inoculation.

6 groups of 14 mice received the following vaccine compositions respectively:
1. pUMVC4 mCCL3 S16A 5 μg+Kolliphor
2. pUMVC4 mCCL4 S16A 5 μg+Kolliphor
3. pUMVC4 mCCL5 S16A 5 μg+Kolliphor
4. pUMVC4 mCCL19 S16A 5 μg+Kolliphor
5. pUMVC4 mXCL1 S16A 5 μg+Kolliphor
6. pUMVC4 5 μg+Kolliphor
7. Untreated

An eight group of naïve mice included 5 animals.

The tetramer assay was carried out as follows:

MHC class I molecules are produced and loaded with a stabilizing peptide that is exchanged with the C22 epitope by exposing the molecules to UV light. The MHC I molecules are multimerized by coupling to fluorescently labelled Streptavidin. To identify neopeptide positive CD8⁺ T cells, cells are co-stained with the multimers and fluorophore conjugated anti-CD3, anti-CD4 and anti-CD8 antibodies. Samples are then analyzed by flow cytometry and the fraction of MHC:C22 positive CD8⁺ is calculated.

To gauge T-cell activation, the following re-stimulation experiment was carried out:

Splenocytes were stimulated with the 5 vaccine-containing neopeptides. In the splenocyte samples, antigen presenting cells process the neopeptides and subsequently present them to T cells, leading to activation of cognate CD4⁺ and CD8⁺ T cells. The activated T cells increase cytokine synthesis, including interferon γ (IFN-γ). IFN-γ producing T cells were detected by ELISpot analysis.

Results

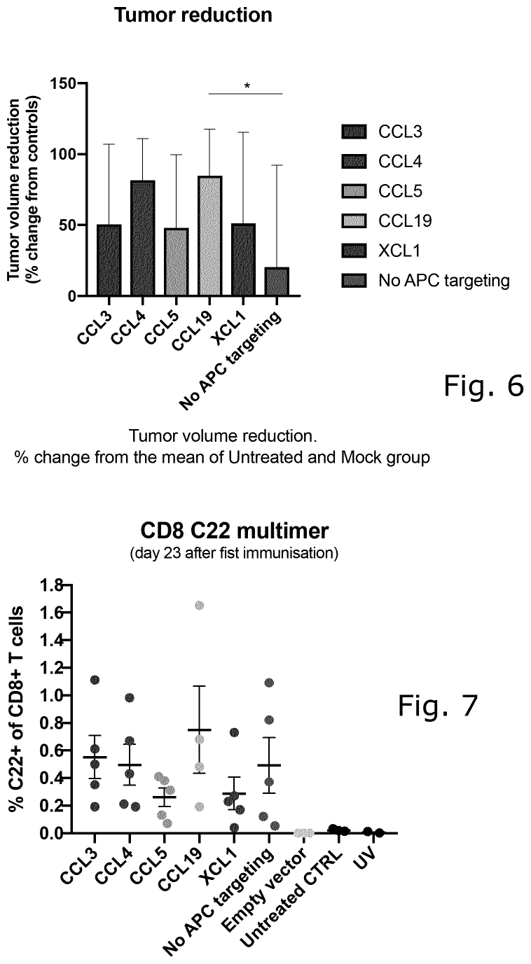

The effect on tumour growth of the immunizations is shown in FIG. 6: Prophylactic immunizations resulted in 50-100% lower tumour volume for mice receiving 5 μg pUMVC4 APC targeting S16A plasmid vector with co-polymers Kolliphor. The tumor reduction was significantly improved in the group treated with 5 μg pUMVC4 mCCL19 S16A with Kolliphor as compared to 5 μg pUMVC4 with no APC targeting S16A with Kolliphor.

Whole blood from all mice where collected at day 7 post tumor inoculation and stained with fluorophore labelled C22 MHC I tetramers. Immunizations with the C22 encoding pUMVC4 APC targeting S16A vaccine induced C22 neo-peptide specific CD8⁺ T cells at high frequencies (average from 0.3 to 0.6 frequency). See FIG. 7.

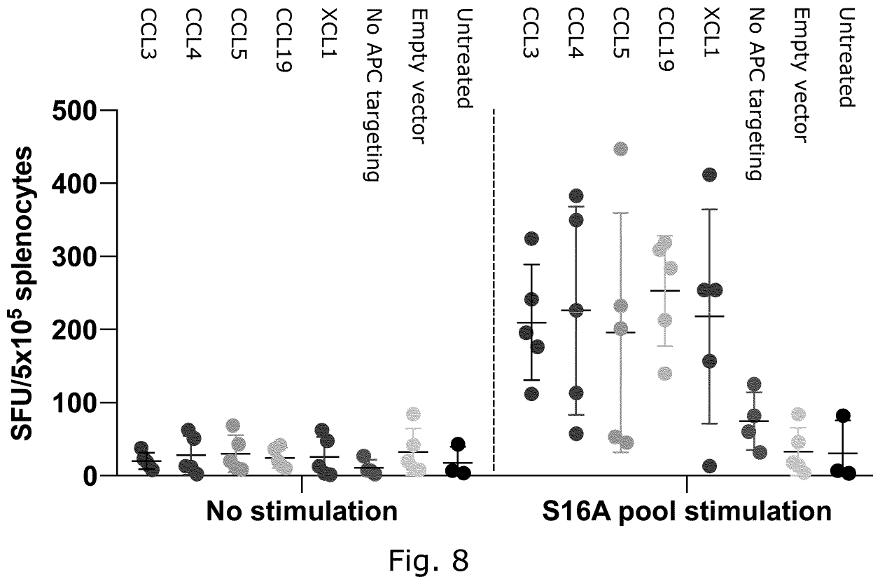

The vaccination with the S16A plasmid induced T cells capable of producing IFNγ in response to subsequent stimulation with neopeptides, whereas samples from animals not immunized with S16A exhibited no cytokine signals upon stimulation with the neopeptides. See FIG. 8.

Further, the DNA vaccines were well-tolerated by the mice; no signs of adverse effects were observed, and the body weight of the mice continuously increased throughout the study, as evident from the increase of body weight change, indicative of healthy and unaffected mice.

Assessment of body weight change for mice immunized with DNA encoding chemokines as APC targeting unit for delivery of neoepitope

| | | |
|---|---|---|
| CCL3 | CCL4 | CCL5 |

| Days | mean | SD | N | mean | SD | N | mean | SD | N |
|---|---|---|---|---|---|---|---|---|---|
| −16 | 0 | 0 | 14 | 0 | 0 | 14 | 0 | 0 | 14 |
| −15 | 0.8863754 | 1.3309119 | 14 | 1.9423182 | 2.6624305 | 14 | 1.2735783 | 2.1555161 | 14 |
| −11 | 1.2574794 | 3.185451 | 14 | 2.1802261 | 3.3544355 | 14 | 4.3486595 | 4.0783625 | 14 |
| −9 | 0.220809 | 4.7964973 | 14 | 0.283442 | 3.0857483 | 14 | 3.0598174 | 4.661057 | 14 |
| −7 | 2.7427221 | 4.0704744 | 14 | 1.7739198 | 3.3477789 | 14 | 3.8801771 | 3.4525704 | 14 |
| −3 | 2.41419 | 4.4013287 | 14 | 2.5137031 | 4.8636941 | 14 | 4.9504809 | 3.8026727 | 14 |
| 1 | 2.3830701 | 4.762877 | 14 | 2.5370244 | 4.4049527 | 14 | 4.2645075 | 4.2478137 | 14 |
| 4 | 0.6451327 | 5.4944956 | 14 | 1.1731022 | 5.3330388 | 14 | 2.6162068 | 4.4934448 | 14 |
| 5 | 2.9150697 | 5.8397319 | 14 | 3.7419904 | 4.0857969 | 14 | 4.9224552 | 4.3104744 | 14 |
| 6 | 3.3658798 | 6.429654 | 14 | 4.5936267 | 3.8893079 | 14 | 5.112862 | 5.3888527 | 14 |
| 8 | 3.3451757 | 5.9851109 | 14 | 4.3506699 | 3.4117179 | 14 | 5.9742509 | 5.3270059 | 14 |
| 11 | 4.9910845 | 5.5779741 | 14 | 4.6701795 | 4.1931341 | 14 | 6.8725523 | 5.2499513 | 14 |
| 12 | 5.1640227 | 6.917586 | 14 | 4.4077461 | 3.7743819 | 14 | 7.702807 | 5.1709529 | 14 |
| 13 | 5.5916725 | 6.15574 | 14 | 5.1101704 | 5.1916002 | 14 | 8.3171774 | 5.4028654 | 14 |
| 15 | 6.2193684 | 7.3421511 | 14 | 6.1394359 | 4.7025327 | 14 | 8.5074718 | 4.6757277 | 14 |
| 18 | 7.2649069 | 7.775726 | 13 | 7.9329011 | 4.1813369 | 13 | 11.858912 | 5.5201394 | 12 |
| 20 | 11.524643 | 7.3146213 | 12 | 8.4188041 | 4.3620684 | 12 | 14.791602 | 5.3405828 | 12 |

| | | |
|---|---|---|
| CCL19 | XCL1 | No APC targeting |

| Days | mean | SD | N | mean | SD | N | mean | SD | N |
|---|---|---|---|---|---|---|---|---|---|
| −16 | 0 | 0 | 14 | 0 | 0 | 14 | 0 | 0 | 14 |
| −15 | 1.4415903 | 3.6817642 | 14 | −0.366308 | 3.2720866 | 14 | 0.8790025 | 1.2659416 | 14 |
| −11 | 1.4575305 | 3.8991463 | 14 | 0.9944677 | 4.1273903 | 14 | 1.957351 | 3.2190635 | 14 |
| −9 | 0.3365991 | 4.3996313 | 14 | 0.2157471 | 3.6052114 | 14 | 1.8717848 | 3.2461756 | 14 |
| −7 | 1.5629255 | 3.6565118 | 14 | 1.1870868 | 2.573715 | 14 | 2.1313298 | 3.1622751 | 14 |
| −3 | 2.0791244 | 4.6838491 | 14 | 2.6463785 | 4.2702206 | 14 | 3.9748831 | 2.3939841 | 14 |
| 1 | 3.1472585 | 6.6713694 | 14 | 3.2033684 | 4.0126726 | 14 | 4.4819436 | 3.1414049 | 14 |
| 4 | 0.8815458 | 6.3940253 | 14 | 1.3979628 | 6.0343284 | 14 | 2.3293464 | 4.337663 | 14 |
| 5 | 2.5304266 | 5.8941714 | 14 | 3.7315569 | 3.77523 | 14 | 4.4258209 | 4.1108208 | 14 |
| 6 | 2.4540823 | 4.7813688 | 14 | 4.8482379 | 3.8677259 | 14 | 4.7680391 | 4.4982046 | 14 |
| 8 | 2.7657444 | 4.6035696 | 14 | 4.43983 | 4.3553127 | 14 | 5.9081958 | 5.2074227 | 14 |
| 11 | 3.951975 | 5.2856939 | 14 | 6.5905758 | 5.3637017 | 14 | 5.0619503 | 4.2023491 | 14 |
| 12 | 5.1074123 | 5.2650377 | 14 | 5.7051446 | 5.4879028 | 14 | 6.3130952 | 3.9546609 | 14 |
| 13 | 5.188319 | 4.7721888 | 14 | 7.93995 | 5.7825594 | 14 | 7.8154602 | 4.4499296 | 14 |
| 15 | 6.266911 | 5.221704 | 14 | 7.8882129 | 5.9709272 | 14 | 8.9640968 | 4.814026 | 14 |
| 18 | 6.3197803 | 4.7643997 | 14 | 9.4112441 | 5.2223158 | 13 | 11.698208 | 3.0616748 | 12 |
| 20 | 7.7426458 | 4.7014022 | 13 | 12.864004 | 6.1921514 | 13 | 12.650733 | 4.3147454 | 11 |

| | Empty plasmid | | | Untreated | | | Naïve mice | | |
|---|---|---|---|---|---|---|---|---|---|
| Days | mean | SD | N | mean | SD | N | mean | SD | N |
| −16 | 0 | 0 | 14 | 0 | 0 | 13 | 0 | 0 | 5 |
| −15 | 1.3411582 | 1.6355957 | 14 | 1.6689002 | 3.3525154 | 13 | −0.118611 | 1.9190125 | 5 |
| −11 | 1.212813 | 2.731037 | 14 | 2.3310089 | 2.3660826 | 13 | −0.162533 | 1.9362329 | 5 |
| −9 | −0.257312 | 2.9411571 | 14 | 1.7733589 | 3.308048 | 13 | 1.3992846 | 3.9184503 | 5 |
| −7 | 0.634383 | 3.618878 | 14 | 2.0655841 | 4.0365386 | 13 | 2.3689432 | 3.7546783 | 5 |
| −3 | 3.0305458 | 4.5913096 | 14 | 3.444122 | 3.8061839 | 13 | 2.7756258 | 2.2299571 | 5 |
| 1 | 2.8390304 | 4.309384 | 14 | 3.5162211 | 4.2468904 | 13 | 3.0060962 | 1.076782 | 5 |
| 4 | 1.4497727 | 4.4667143 | 14 | −0.245309 | 7.7964932 | 13 | −0.249337 | 3.2368439 | 5 |
| 5 | 3.5042952 | 4.5394432 | 14 | 3.7433095 | 5.1731233 | 13 | 2.1795295 | 1.2681 | 5 |
| 6 | 3.7676028 | 3.7914055 | 14 | 4.422164 | 5.9939937 | 13 | 2.9423582 | 1.3548533 | 5 |
| 8 | 4.0142092 | 4.0396446 | 14 | 5.0562335 | 6.120553 | 13 | 3.4004358 | 1.1941085 | 5 |
| 11 | 6.27957 | 4.0380831 | 14 | 4.8483895 | 5.9276173 | 13 | 4.4244347 | 1.5398234 | 5 |
| 12 | 6.9780764 | 5.7678588 | 14 | 5.8418009 | 5.4677348 | 13 | 5.9894487 | 0.9256987 | 5 |
| 13 | 7.1979332 | 4.0540421 | 14 | 6.6638885 | 6.4712168 | 13 | 6.760866 | 1.752727 | 5 |
| 15 | 8.5884406 | 5.9671708 | 14 | 7.0848026 | 7.4386101 | 13 | 9.3695385 | 2.843451 | 5 |
| 18 | 10.358708 | 6.3822571 | 14 | 9.3929114 | 7.7285807 | 10 | 8.7600563 | 3.8239072 | 4 |
| 20 | 11.427638 | 5.4351893 | 9 | 8.7648269 | 8.578872 | 7 | 7.2360458 | 6.5963891 | 5 |

Conclusions

The Kolliphor delivered pUMVC4 plasmid vectors containing different APC targeting units and the S16A neoepitopes resulted in CT26 anti-tumour effects. A dose as low as 5 μg of DNA resulted in highly significantly tumour volume AUC reduction compared to control groups, demonstrating the high efficacy of the APC targeting DNA vaccine. S16A neopeptide re-stimulation showed similar T cell immunogenicity profiles in splenocytes across groups that received S16A Plasmid vector independent on the APC targeting unit.

The vaccines were well-tolerated by the mice; no signs of adverse effects were observed, and the body weight of the mice continuously increased throughout the study, indicative of healthy and unaffected mice.

### Example 2

### Assessment of Other Chemokines, Cytokines, Fv Fragments and Peptides as APC Targeting Unit for Delivery of Neoepitopes

In this study we wished to test whether other chemokines and cytokines as well as APC targeting molecules outside the chemokine family, such as antibodies recognizing receptors on the APC or small peptide ligands binding to the surface receptors of the APC were able to induce neo-peptide specific T cells and reduce tumor growth to the same extend as seen in the previous study. Furthermore, we wished to monitor the impact of the vaccine on the well-being of vaccinated mice.

Plasmids for DNA vaccination were based on the commercially available pUMVC4™ vector available from Aldevron.

Eight expression vectors generated with the pUMVC4 as backbone and an APC targeting unit guiding the neoepitopes in S16A was constructed. The various chemokines, Fv fragments and peptide ligands used as APC targeting units are listed in the following table:

| APC targeting unit | SEQUENCE ID NO |
|---|---|
| mCCL19 | SEQ ID NO: 39 |
| mCCL20 | SEQ ID NO: 41 |
| mCCL21 | SEQ ID NO: 42 |
| mGM-CSF | SEQ ID NO: 43 |
| Anti-mDEC-205 Fv fragment | SEQ ID NO: 45 |
| Anti-mCLEC-9 Fv fragment | SEQ ID NO: 46 |
| CLEC9 ligand | SEQ ID NO: 47 |
| FLT3 ligand | SEQ ID NO: 48 |

Study Plan

Mice received immunizations with the test vaccines on days −15, −8, −1, 6, and 13 relative to the CT26 tumour inoculation on day 0. Each immunization consisted of injection of 50 μl vaccine in the left and right tibia, respectively. Blood samples for C22 MHC I testing in a tetramer assay were obtained from the test animals on day −2 and 8 after inoculation.

9 groups of 13 mice received the following vaccine compositions respectively:
1. pUMVC4 mCCL19 S16A 5 μg+Kolliphor
2. pUMVC4 (empty) 5 μg+Kolliphor
3. pUMVC4 anti mCLEC9 Fv S16A 5 μg+Kolliphor
4. pUMVC4 mCLEC-9 ligand S16A 5 μg+Kolliphor
5. pUMVC4 mCCL20 S16A 5 μg+Kolliphor
6. pUMVC4 mCCL21 S16A 5 μg+Kolliphor
7. pUMVC4 anti mDEC-205 Fv S16A 5 μg+Kolliphor
8. pUMVC4 mFLT3L S16A 5 μg+Kolliphor
9. pUMVC4 mGM-CSF S16A 5 μg+Kolliphor

A tenth group of naïve mice included 5 animals.

Read-outs of the experiment were tumour volume, measurement of neo-epitope-specific CD8⁺ T cells in circulation and functional neo-epitope-specific T cells isolated from spleens.

Results

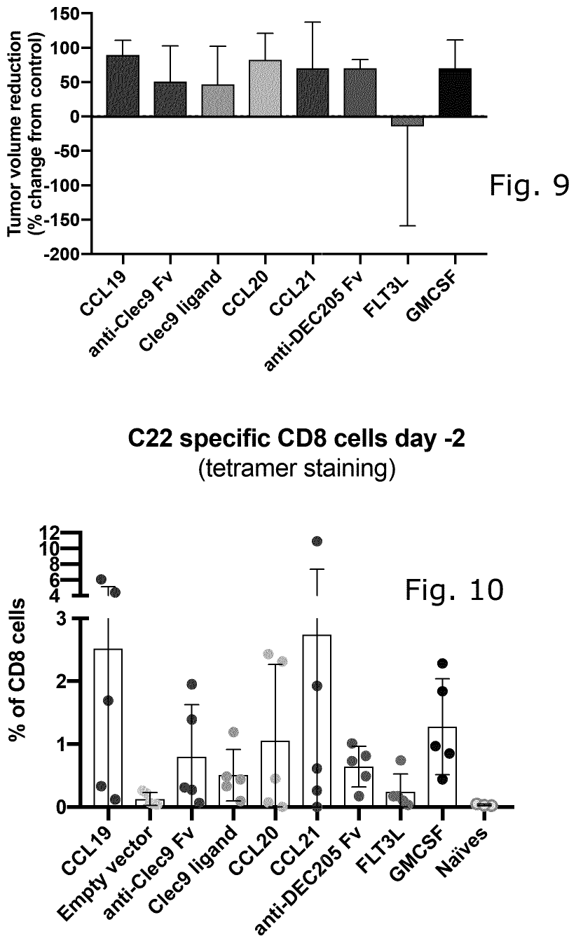

The effect on tumour growth of the immunizations is shown in FIG. 9: Prophylactic immunizations resulted in 50-100% tumor size reduction for mice receiving 5 μg pUMVC4 APC targeting S16A plasmid vector with co-polymers Kolliphor compared to 5 μg pUMVC4 with Kolliphor.

Whole blood from all mice where collected at day −2 and stained with fluorophore labelled C22 MHC I tetramers. Immunizations with the C22 encoding pUMVC4 APC targeting S16A vaccine induced C22 neo-peptide specific CD8⁺ T cells at high frequencies (average from 0.3 to 0.6 frequency). See FIG. 10. The effect was markedly better than the control empty vector (average under 0.1 frequency) without neoepitopes.

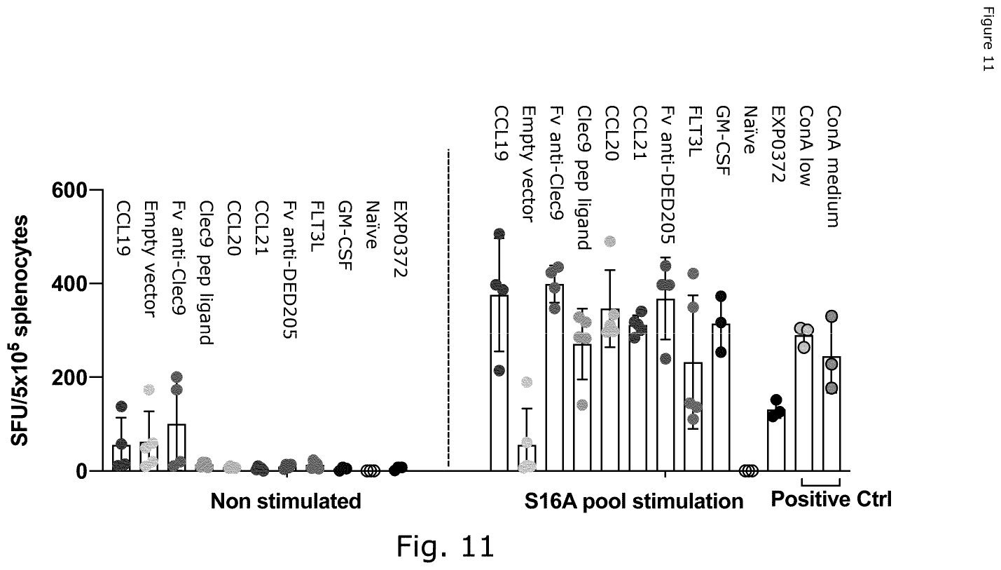

At endpoint vaccination with all the S16A containing plasmids induced T cells derived from spleen that were capable of producing IFNγ in response to subsequent stimulation with neo-peptides. Samples from animals that were not immunized with S16A (empty plasmid and naïve mice) exhibited no cytokine signals upon stimulation with the neo-peptides. See FIG. 11.

Further, the DNA vaccines were well-tolerated by the mice; no signs of adverse effects were observed, and the body weight of the mice continuously increased throughout the study, as evident from the increase on body weight change, indicative of healthy and unaffected mice.

Assessment of body weight change for mice immunized with DNA encoding Fv fragments and peptides as APC targeting unit for delivery of neoepitopes.

| | CCL19 | | | Empty vector | | | Fv anti-Clec9 | | | Clec9 pep ligand | | |
|---|---|---|---|---|---|---|---|---|---|---|---|---|
| Days | mean | SD | N | mean | SD | N | mean | SD | N | mean | SD | N |
| −15 | 0 | 0 | 13 | 0 | 0 | 8 | 0 | 0 | 13 | 0 | 0 | 13 |
| −14 | 1.139148 | 1.353041 | 13 | 0.7274891 | 1.6277915 | 8 | 1.0104646 | 2.2013327 | 13 | 1.4586595 | 1.9248236 | 13 |
| −12 | 1.9608584 | 2.8363333 | 13 | 0.076736 | 2.0535941 | 8 | 0.7951009 | 2.8320476 | 13 | 2.1721557 | 2.942616 | 13 |
| −8 | 4.8871238 | 3.3673985 | 13 | 2.9945308 | 3.5834629 | 8 | 2.4139883 | 4.3354556 | 13 | 4.27686 | 4.2014545 | 13 |
| −5 | 3.993838 | 2.417825 | 13 | 3.3228029 | 2.966784 | 8 | 6.0817445 | 3.1310146 | 13 | 5.012723 | 3.5305563 | 13 |
| −1 | 2.5248411 | 1.7932877 | 13 | 4.8718416 | 2.8314056 | 8 | 4.2528719 | 3.0745385 | 13 | 4.7154367 | 2.5850021 | 13 |
| 2 | 6.0475665 | 2.4763482 | 13 | 7.2299469 | 1.5077816 | 8 | 6.5465987 | 3.2364261 | 13 | 7.6309345 | 2.5155128 | 13 |
| 5 | 7.4167472 | 2.8104202 | 13 | 8.1439476 | 2.9839372 | 8 | 7.8874323 | 3.2500325 | 13 | 7.8902886 | 2.9573388 | 13 |
| 7 | 8.0583294 | 3.2876568 | 13 | 10.222268 | 3.6128255 | 8 | 9.0977444 | 3.9930344 | 13 | 8.3282824 | 2.6349014 | 13 |
| 9 | 7.8405825 | 3.8765573 | 13 | 9.5793539 | 3.052331 | 8 | 8.9420401 | 3.6235839 | 13 | 8.8672662 | 2.9374285 | 13 |
| 12 | 9.3909254 | 2.8600445 | 13 | 10.344554 | 4.3233427 | 8 | 10.068073 | 5.2140401 | 13 | 10.69861 | 3.2917778 | 13 |
| 14 | 8.0597907 | 2.7665782 | 13 | 10.881072 | 3.9925636 | 8 | 9.8156458 | 3.7635493 | 13 | 9.818513 | 2.8993949 | 13 |
| 16 | 9.6136928 | 2.6133819 | 13 | 14.541798 | 5.6578245 | 8 | 12.050101 | 2.9271745 | 12 | 11.801434 | 3.2232206 | 13 |
| 19 | 10.352757 | 3.5465027 | 12 | 15.176842 | 5.9073788 | 7 | 13.363635 | 4.0756288 | 11 | 11.143421 | 3.7802431 | 13 |

| | CCL20 | | | CCL21 | | | Fv anti-DEC205 | | |
|---|---|---|---|---|---|---|---|---|---|
| Days | mean | SD | N | mean | SD | N | mean | SD | N |
| −15 | 0 | 0 | 13 | 0 | 0 | 13 | 0 | 0 | 13 |
| −14 | 0.6307569 | 2.4728171 | 13 | 1.3543557 | 1.7192062 | 13 | 0.6293231 | 1.9084697 | 13 |
| −12 | 2.1195786 | 3.2126495 | 13 | 1.8293204 | 1.8917054 | 13 | 2.100173 | 3.1837618 | 13 |
| −8 | 4.6943438 | 2.9687584 | 13 | 4.3742356 | 2.9070852 | 13 | 4.1636965 | 2.5322817 | 13 |
| −5 | 4.3350389 | 1.9419675 | 13 | 5.5359249 | 2.966961 | 13 | 4.5458723 | 2.2127792 | 13 |
| −1 | 3.9570983 | 1.9996787 | 13 | 4.3963027 | 2.8908727 | 13 | 3.8951226 | 2.6855639 | 13 |
| 2 | 7.8824803 | 3.9559045 | 13 | 5.3131697 | 2.8459311 | 13 | 6.5721784 | 3.5730706 | 13 |
| 5 | 8.7628661 | 3.7619537 | 13 | 7.4036597 | 5.122963 | 13 | 7.1031392 | 3.9039116 | 13 |
| 7 | 11.224134 | 1.9098859 | 13 | 9.7279721 | 3.3447985 | 13 | 9.7004289 | 3.8788376 | 13 |
| 9 | 9.7266611 | 3.6525425 | 13 | 9.4743827 | 3.7748095 | 13 | 8.7800297 | 5.5312064 | 13 |
| 12 | 11.340991 | 2.9263815 | 13 | 9.7580625 | 2.6018417 | 13 | 11.043509 | 3.6359454 | 13 |
| 14 | 9.742928 | 3.7752266 | 13 | 19.6435734 | 2.4700711 | 13 | 10.085142 | 3.0395913 | 13 |
| 16 | 11.899696 | 3.4063403 | 13 | 11.569268 | 3.3297408 | 13 | 11.596663 | 2.8781698 | 12 |
| 19 | 12.246312 | 3.8715511 | 13 | 11.66981 | 3.477578 | 13 | 11.882038 | 2.5270066 | 12 |
| | FLT3L | | | GM-CSF | | | Naïve | | |
| Days | mean | SD | N | mean | SD | N | mean | SD | N |
| −15 | 0 | 0 | 13 | 0 | 0 | 13 | 0 | 0 | 4 |
| −14 | 1.6487397 | 3.0170806 | 13 | 1.5531263 | 1.7891121 | 13 | −0.563251 | 0.8442212 | 4 |
| −12 | 3.1594828 | 3.6284443 | 13 | 3.0322935 | 1.4896749 | 13 | −0.803813 | 1.7905142 | 4 |
| −8 | 6.4052386 | 5.152845 | 13 | 5.1544162 | 4.6237373 | 13 | 3.3374585 | 2.5580524 | 4 |
| −5 | 6.4713217 | 4.1792643 | 13 | 8.29216 | 3.3715389 | 13 | 6.0071692 | 4.7847333 | 4 |
| −1 | 4.9651132 | 2.2349646 | 12 | 5.8838183 | 2.5453088 | 13 | 3.096435 | 1.3922387 | 4 |
| 2 | 7.8271327 | 3.7384128 | 12 | 7.6394922 | 3.0395695 | 13 | 5.967283 | 1.5412159 | 4 |
| 5 | 8.8922992 | 3.8481706 | 12 | 8.6807205 | 3.2160132 | 12 | 6.4137115 | 1.5640278 | 4 |
| 7 | 9.8318543 | 4.8462709 | 12 | 10.390035 | 3.8090331 | 12 | 7.6586037 | 2.8712901 | 4 |
| 9 | 9.5842145 | 3.5666071 | 12 | 9.9943916 | 3.9615974 | 12 | 8.6974545 | 0.9864127 | 4 |
| 12 | 11.190934 | 4.0935224 | 12 | 12.309059 | 4.5705428 | 12 | 9.522338 | 2.2707586 | 4 |
| 14 | 11.369933 | 3.6257843 | 12 | 10.605977 | 3.3638856 | 12 | 10.327501 | 2.7798875 | 4 |
| 16 | 14.019179 | 5.0670855 | 12 | 13.42857 | 3.6152313 | 12 | 11.444177 | 2.2889767 | 4 |
| 19 | 13.274705 | 2.505272 | 10 | 13.055231 | 3.2021551 | 12 | 9.7444706 | 2.2332287 | 4 |

Conclusions

The Kolliphor delivered pUMVC4 plasmid vectors containing different APC targeting units and the S16A neoepitopes resulted in CT26 anti-tumour effects and circulating C22 neoepitope specific CD8+ T cells. A dose as low as 5 μg of DNA resulted in tumour volume reduction compared to control groups, demonstrating the high efficacy of the APC targeting DNA vaccine. S16A neo-peptide re-stimulation showed similar T cell immunogenicity profiles in splenocytes across groups that received S16A Plasmid vector independent on the APC targeting unit. Furthermore, the DNA vaccines were well-tolerated by the mice as assessed by body weight change during the experiment.

### Example 3

Assessment of Various Multimerization Units for the APC Targeting Technology for Delivery of Neoepitopes

In this study we wished to test whether an APC targeting neoepitope vaccine with multimerization units other than a fragment from human IgG3, such as other immuno globulins (Ig), synthetic proteins or collagen fragments, were able to induce neo-peptide specific T cells and reduce tumor growth. Murine CCL19 was used as APC targeting unit. Furthermore, we wished to monitor the impact of the vaccine on the well-being of vaccinated mice

Plasmids for DNA vaccination were based on the commercially available pUMVC4™ vector available from Aldevron.

Five new expression vectors generated with the pUMVC4 as backbone and CCL19 as APC targeting unit guiding the neoepitopes in S16A were constructed. The various multimerization units used for the various constructs are listed in the table below:

| Molecule | Function | SEQ ID NO: |
|---|---|---|
| GS linker (for monomer) | No | SEQ ID NO: 13 |
| | multimerization | |
| dHLX protein | dimerization | SEQ ID NO: 49 |
| hMHD2 (human IgM) | dimerization | SEQ ID NO: 51 |
| Collagen XVIII trimerisation | Trimerization | SEQ ID NO: 52 |
| domain | | |
| p53 synthetic protein | Tetramerization | SEQ ID NO: 53 |

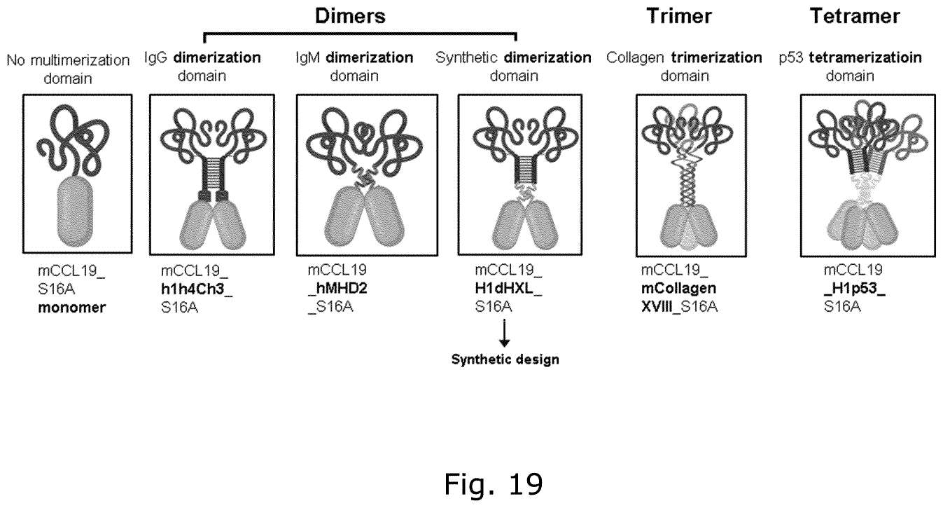

The new DNA constructs were compared to mCCL19 H1H4CH3 S16A construct with IgG3 dimerization domain (SEQ ID NO: 59, 60, and 34). See overview in FIGS. 18 and 19.

Study Plan

Mice received immunizations with the test vaccines on days −14, −7, 1, 8, and 15 relative to the CT26 tumour inoculation on day 0. Each immunization consisted of injection of 50 μl vaccine in the left and right tibialis anterior, respectively. Blood samples for C22 MHC I testing in a tetramer assay were obtained from the test animals on day 7 after inoculation.

7 groups of 13 mice received the following vaccine compositions respectively:
1. pUMVC4 (empty) 5 μg+Kolliphor
2. pUMVC4 mCCL19 S16A monomer 5 μg+Kolliphor
3. pUMVC4 mCCL19 H1H4CH3 S16A 5 μg+Kolliphor
4. pUMVC4 mCCL19 H1dHLX S16A 5 μg+Kolliphor
5. pUMVC4 mCCL19 H1p53 S16A 5 μg+Kolliphor
6. pUMVC4 mCCL19 hMHD2 S16A 5 μg+Kolliphor
7. pUMVC4 mCCL19 mCollagenXVIII S16A 5 μg+Kolliphor

An eighth group of naïve mice included 4 animals.

Read-outs of the experiment were body weight change relative to the weight at the first immunization, tumour volume reduction, measurement of neo-epitope-specific CD8⁺ T cells in circulation and functional neo-epitope-specific T cells isolated from spleens.

Results

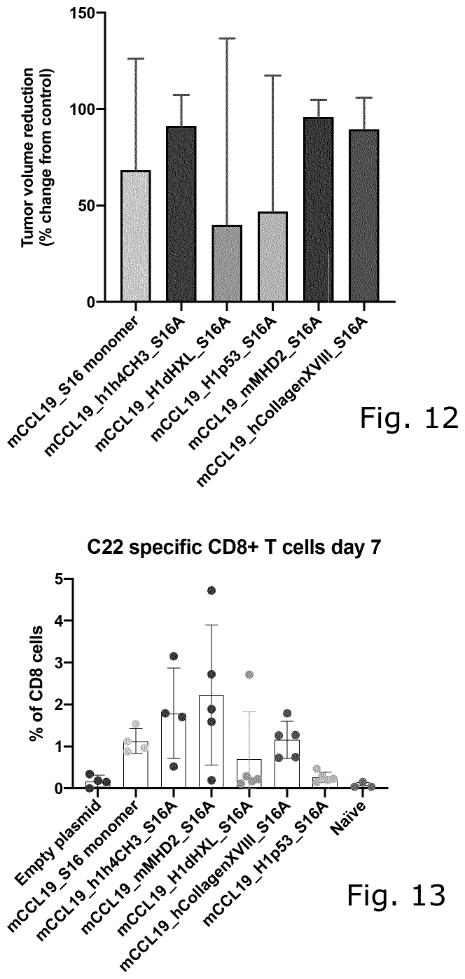

The effect on tumour growth of the immunizations is shown in FIG. 12: Prophylactic immunizations resulted in 50-100% tumor size reduction for all groups of mice receiving 5 μg plasmid vector pUMVC4 mCCL19 containing the neoepitopes S16A in combination with Kolliphor compared to 5 μg empty plasmid with Kolliphor. The groups receiving a DNA design with a multimerization unit that was of Ig origin or collagen performed the best.

Whole blood from all mice where collected at day 7 and stained with fluorophore labelled C22 MHC I tetramers. Immunizations with the C22 encoding pUMVC4 CCL19 S16A vaccine all gave rise to a detectable level of C22 neo-peptide specific CD8⁺ T cells (average ranging from 0.3 to 5% frequency). See FIG. 13. The effect was most pronounced for the IgG containing designs (H1H4CH3 and hMHD2, average around 2% frequency), medium response for the monomer and the trimer (monomer and hCollagenXVIII, average over 1% frequency), which were markedly better than the synthetic protein designs (H1dHLX and H1p53, average below 0.5% frequency) and the empty plasmid control.

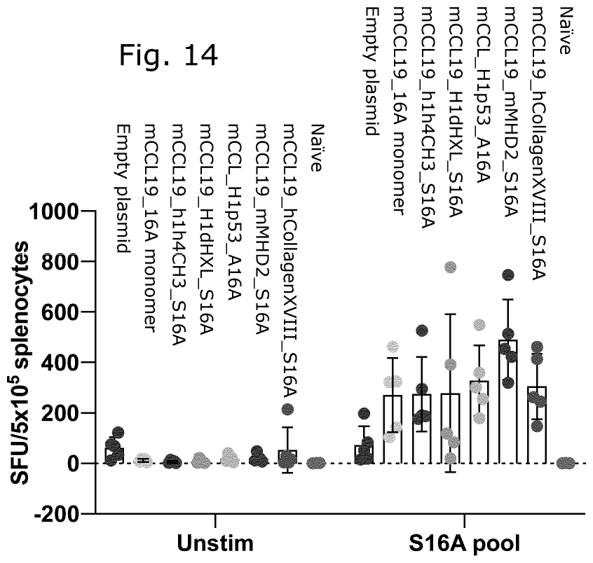

At endpoint vaccination with the all the S16A containing plasmids induced T cells derived from spleen that were capable of producing IFNγ in response to subsequent stimulation with neo-peptides. Samples from animals that were not immunized with S16A (empty plasmid and naïve) exhibited no cytokine signals upon stimulation with the neo-peptides. See FIG. 14.

Further, the DNA vaccines were well-tolerated by the mice; no signs of adverse effects were observed, and the body weight of the mice continuously increased throughout the study, as evident from the increase in body weight change, indicative of healthy and unaffected mice.

Assessment of body weight change for mice immunized with DNA encoding various multimerization units for the APC targeting technology for delivery of neoepitopes

| | Empty plasmid | | | mCCL19_S16 monomer | | | mCCL19_h1h4CH3_S16A | | | mCCL19_H1dHXL_S16A | | |
|---|---|---|---|---|---|---|---|---|---|---|---|---|
| Days | mean | SD | N | mean | SD | N | mean | SD | N | mean | SD | N |
| −14 | 0 | 0 | 15 | 0 | 0 | 14 | 0 | 0 | 15 | 0 | 0 | 14 |
| −13 | 1.8065903 | 1.4658816 | 15 | 1.6868882 | 2.0734565 | 14 | 2.051628 | 1.9325912 | 15 | 2.5402176 | 1.3812768 | 14 |
| −10 | 3.5160765 | 2.1254483 | 15 | 4.6467974 | 2.9015154 | 14 | 4.6245979 | 2.0965962 | 15 | 4.0723021 | 2.1302442 | 14 |
| −7 | 3.770133 | 2.0686022 | 15 | 4.8105898 | 4.0003457 | 14 | 5.4134208 | 2.8454668 | 15 | 4.9851396 | 1.9122281 | 14 |
| −5 | 6.628435 | 3.010984 | 15 | 7.1382895 | 4.7086045 | 14 | 9.0138749 | 3.3928789 | 15 | 7.5185625 | 3.0997695 | 14 |
| −3 | 7.0289117 | 2.4257649 | 15 | 7.0974051 | 4.3724483 | 14 | 9.1890066 | 3.1346121 | 15 | 7.619037 | 3.197423 | 14 |
| 0 | 7.3468783 | 3.4857441 | 15 | 8.3409671 | 3.9141863 | 14 | 8.4347524 | 2.1744504 | 15 | 6.5620459 | 2.8396834 | 14 |
| 1 | 4.5328145 | 3.6591673 | 15 | 5.4788081 | 4.116406 | 14 | 6.6809992 | 3.0757316 | 15 | 5.0012801 | 3.6445776 | 14 |
| 2 | 5.9201369 | 3.5739631 | 15 | 8.0948364 | 5.1165168 | 14 | 8.8516984 | 3.5081711 | 15 | 4.8054743 | 3.5353776 | 14 |
| 4 | 6.85005 | 3.4916068 | 15 | 9.0792098 | 3.9091538 | 14 | 10.529619 | 3.8993072 | 15 | 6.6966493 | 4.0431882 | 14 |
| 7 | 7.1445987 | 2.9992626 | 15 | 11.510528 | 6.0430493 | 14 | 11.210636 | 4.2788492 | 15 | 7.6657322 | 4.5257821 | 14 |
| 9 | 10.542385 | 4.6710614 | 15 | 10.840238 | 3.6489439 | 14 | 13.746003 | 5.5857997 | 15 | 10.329819 | 4.839032 | 14 |
| 11 | 10.462602 | 3.9313322 | 15 | 12.058571 | 2.3601854 | 14 | 14.688285 | 6.4618683 | 15 | 9.9963472 | 3.7006016 | 14 |
| 14 | 13.315998 | 4.5686599 | 15 | 13.551067 | 3.7850659 | 14 | 14.282771 | 5.9143999 | 15 | 10.760224 | 3.9935569 | 13 |
| 16 | 13.755865 | 4.6056887 | 14 | 14.472833 | 5.4109189 | 13 | 15.354434 | 5.5457766 | 15 | 13.055214 | 3.9912675 | 13 |
| 18 | 15.261076 | 3.7202453 | 14 | 16.056548 | 5.4333788 | 12 | 17.775144 | 7.3666753 | 14 | 14.707669 | 5.3675715 | 12 |

| | mCCL19_H1p53_S16A | | | mCCL19_mMHD2_S16A | | | mCCL19_hCollagenXVIII_S16A | | | Naïve | | |
|---|---|---|---|---|---|---|---|---|---|---|---|---|
| Days | mean | SD | N | mean | SD | N | mean | SD | N | mean | SD | N |
| −14 | 0 | 0 | 14 | 0 | 0 | 14 | 0 | 0 | 14 | 0 | 0 | 4 |
| −13 | 1.7149315 | 2.2405615 | 14 | 1.0994299 | 1.7883377 | 14 | 1.638233 | 1.4133702 | 14 | 1.0119102 | 0.9299167 | 4 |
| −10 | 5.21012 | 3.4689379 | 14 | 3.0213857 | 2.5928036 | 14 | 4.1856776 | 2.6809016 | 14 | 5.3438235 | 1.4548287 | 4 |
| −7 | 4.7338799 | 4.3510254 | 14 | 4.2950687 | 2.9643819 | 14 | 4.4349953 | 2.7134899 | 14 | 5.150205 | 3.0529469 | 4 |
| −5 | 6.0298098 | 4.0751979 | 14 | 6.7843427 | 2.9550719 | 14 | 6.7596373 | 2.4500964 | 14 | 6.7241385 | 2.8091057 | 4 |
| 3 | 6.3863026 | 3.5903038 | 14 | 7.020561 | 3.1511815 | 14 | 7.2101461 | 3.3531306 | 14 | 6.6671358 | 3.0599535 | 4 |
| 0 | 5.0115533 | 3.4082094 | 14 | 4.0979031 | 5.4476714 | 14 | 8.1756837 | 4.4501707 | 14 | 6.8818255 | 3.1676591 | 4 |
| 1 | 4.1558597 | 3.3628039 | 14 | 3.2625642 | 3.8224016 | 14 | 5.5607829 | 4.9334063 | 14 | 3.4020269 | 2.5567755 | 4 |
| 2 | 4.6349486 | 3.6688398 | 14 | 4.0306469 | 2.8672514 | 14 | 7.211852 | 4.228828 | 14 | 5.0527072 | 3.859303 | 4 |
| 4 | 6.5396079 | 3.8835809 | 14 | 6.1143302 | 2.917494 | 14 | 8.3746865 | 5.0902167 | 14 | 7.3629684 | 2.1437738 | 4 |
| 7 | 8.2979131 | 3.4064541 | 14 | 7.2981185 | 3.7648275 | 14 | 8.7555461 | 4.1548737 | 14 | 8.0605716 | 3.2966171 | 4 |
| 9 | 10.093287 | 3.6543979 | 14 | 11.022503 | 4.1735587 | 14 | 10.427133 | 4.2394149 | 14 | 10.407827 | 3.1963835 | 4 |
| 11 | 11.298379 | 4.1873395 | 14 | 11.46006 | 5.5782851 | 14 | 11.462716 | 3.88127 | 14 | 11.031481 | 3.5252434 | 4 |
| 14 | 12.696664 | 3.7677507 | 14 | 12.026869 | 5.2600742 | 14 | 11.312763 | 3.9910123 | 14 | 11.057459 | 5.3188489 | 4 |
| 16 | 13.333518 | 4.8120939 | 14 | 12.579142 | 4.4309364 | 13 | 11.480441 | 4.5051312 | 11 | 12.035978 | 4.2855628 | 4 |
| 18 | 14.617345 | 3.6781525 | 13 | 14.078862 | 5.3951134 | 11 | 13.006091 | 3.3628816 | 11 | 16.391946 | 5.8096504 | 3 |

Conclusions

The Kolliphor delivered pUMVC4 plasmid vectors containing mCCL19, different multimerization units and the S16A neoepitopes resulted in CT26 anti-tumour effects from 50-100%. The groups receiving a DNA design with a multimerization unit that was of Ig origin or collagen performed the best. The neoepitope specific CD8+ T cell response at day 7 corresponded to the antitumor effects and the level of functional T cells at endpoint (as measured by IFNγ secretion) was comparable between groups receiving a DNA design containing neoepitopes. Furthermore, the DNA vaccines were well-tolerated by the mice as assessed by body weight change during the experiment.

### Example 4

### Assessment of Combination/Dissociation of the Separate Units for the APC Targeting Technology for Delivery of Neoepitopes

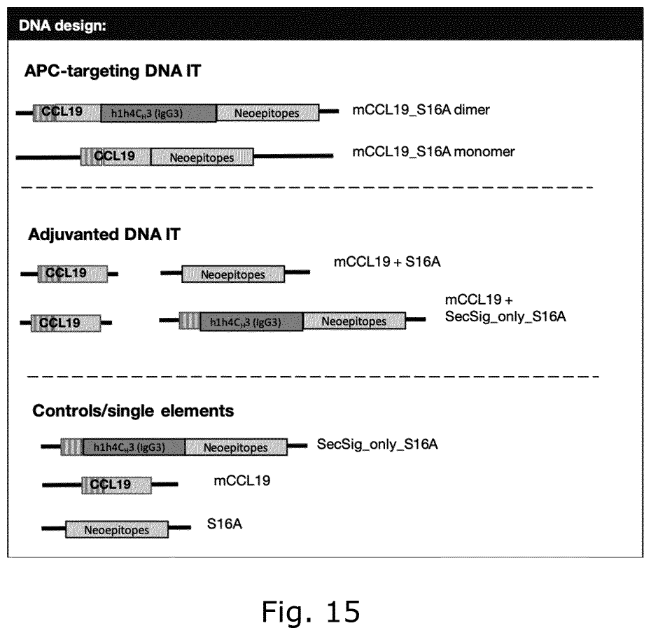

In this study we wished to test whether all three modules; APC targeting, multimerization domain and neoepitopes, needed to be physically linked together as a fusion protein product to perform optimally, or if the mere presence of the chemokine as an adjuvant is sufficient as well as if the dimer/secretion of the neoepitopes ads to the anti-tumor effect and T cell response for the APC targeting neoepitope vaccine. Murine CCL19 was used as chemokine for all DNA constructs. See overview in FIG. 15.

Plasmids for DNA vaccination were based on the commercially available pUMVC4™ vector available from Aldevron.

Three new expression vectors were generated with the pUMVC4 as backbone containing either mCCL19 alone (SEQ ID NO: 39), the neoepitopes S16A alone or the secretion signal (SecSig), the dimerization domain H1H3CH3 of IgG3 (SEQ ID NO: 59, 60 and 34) and S16A neoepitopes. Listed in FIG. 15.

The new DNA constructs, and combinations hereof, were compared to mCCL19 H1H4CH3 S16A construct encoding the fusion protein (referred to as mCCL19 S16A dimer below) as well as the mCCL19 S16A monomer.

Study Plan

Mice received immunizations with the test vaccines on days −14, −7, 1, 8, and 15 relative to the CT26 tumour inoculation on day 0. Each immunization consisted of injection of 50 μl vaccine in the left and right tibialis anterior, respectively. Blood samples for C22 MHC I testing in a tetramer assay were obtained from the test animals on day 9 after inoculation.

8 groups of 13 mice received the following vaccine compositions respectively:
1. pUMVC4 (empty) 10 μg+Kolliphor
2. pUMVC4 mCCL19 S16A dimer 5 μg+Kolliphor
3. pUMVC4 mCCL19 S16A monomer
4. pUMVC4 mCCL19 5 μg+pUMVC4 S16A 5 μg+Kolliphor
5. pUMVC4 mCCL19 5 μg+pUMVC4 SecSig H1H4CH3 S16A 5 μg+Kolliphor
6. pUMVC4 SecSig H1H4CH3 S16A 5 μg+Kolliphor
7. pUMVC4 mCCL19 5 μg+Kolliphor
8. pUMVC4 S16A 5 μg+Kolliphor

A ninth group of naïve mice included 4 animals.

Read-outs of the experiment were body weight change relative to the weight at the first immunization, tumour volume, and measurement of neo-epitope-specific CD8⁺ T cells in circulation.

Results

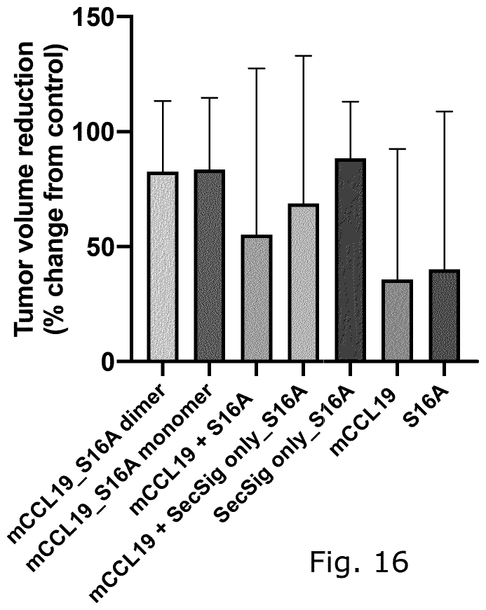

The effect on tumour growth of the immunizations is shown in FIG. 16: Prophylactic immunizations resulted in tumor size reduction for all groups of mice receiving mCCL19 and neoepitopes, independent of if the components are encoded as a fusion protein or as separate protein products. The non-targeted fusion protein comprised of SecSig, H1H4CH3 dimerization domain and neoepitopes also had significant antitumor effect, indicating that secretion and dimerization of the neoepitopes improves the antitumor effect.

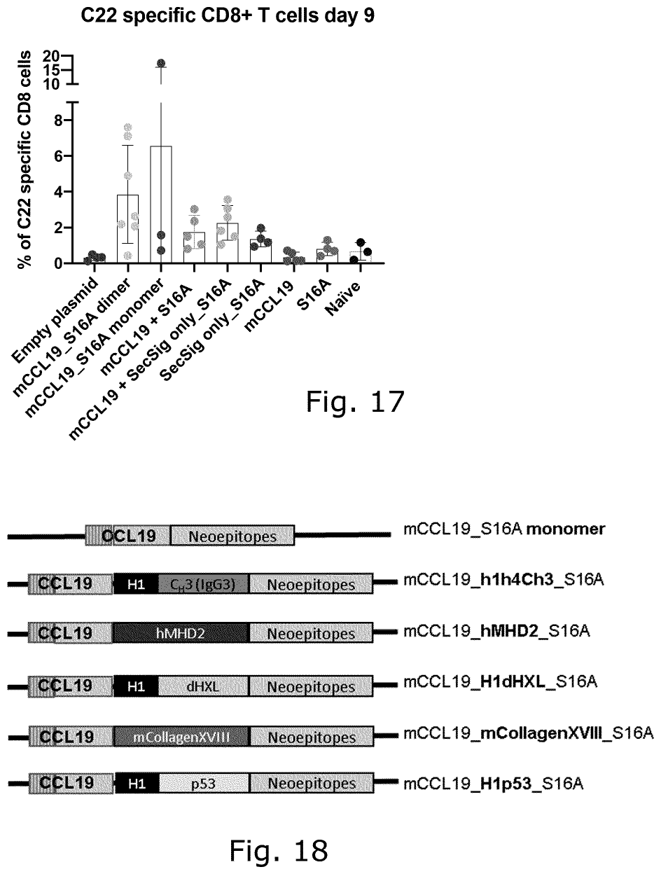

Whole blood from all mice where collected at day 9 and stained with fluorophore labelled C22 MHC I tetramers. Immunizations with all vaccines containing the epitope C22, which is part of S16A, gave rise to a detectable level of C22 neo-peptide specific CD8⁺ T cells (average ranging from 0.5 to 15% frequency). See FIG. 17. The effect was most pronounced for the fusion protein designs (mCCL19 S16A dimer and mCCL19 S16A monomer, average around 4% frequency), whereas the combination of the individual mCCL19 product with the non-targeted fusion protein comprised of SecSig, H1H4CH3 dimerization domain and neoepitopes also gave rise to high T cell levels (average over 1.5% frequency), which were markedly better than the effect of the single components (mCCL19 and S16A, average below 0.5% frequency) and the empty plasmid control.

Further, the DNA vaccines were well-tolerated by the mice; no signs of adverse effects were observed, and the body weight of the mice continuously increased throughout the study, as evident from the increase in body weight change, indicative of healthy and unaffected mice.

Assessment of body weight change for mice immunized with DNA encoding combination/dissociation of the separate units for the APC targeting technology for delivery of neoepitopes

| | | |
|---|---|---|
| Empty plasmid | mCCL19 S16A dimer | mCCL19 S16A monomer |

| Days | mean | SD | N | mean | SD | N | mean | SD | N |
|---|---|---|---|---|---|---|---|---|---|
| −14 | 0 | 0 | 13 | 0 | 0 | 13 | 0 | 0 | 13 |
| −13 | 0.2479374 | 1.885907 | 13 | 0.8746918 | 2.0789501 | 13 | 0.1278129 | 2.3391707 | 13 |
| −10 | 0.2764575 | 3.6292702 | 13 | −1.438595 | 2.359862 | 13 | −0.252501 | 2.9117128 | 13 |
| −7 | 4.0769331 | 2.9879401 | 13 | 1.151358 | 3.1574658 | 13 | 3.5386661 | 2.6480586 | 13 |
| −5 | 4.5127835 | 2.8127309 | 13 | 1.0586818 | 3.412296 | 13 | 4.1373635 | 3.2804711 | 13 |
| −3 | 4.766386 | 2.3286143 | 13 | 0.6268969 | 3.0403871 | 13 | 3.7652486 | 3.7242678 | 13 |
| 0 | 5.5395726 | 2.8854379 | 13 | 2.1639226 | 2.906687 | 13 | 5.4709868 | 3.1712732 | 13 |
| 1 | 6.8068808 | 3.0850282 | 13 | 2.8658581 | 3.4593078 | 13 | 6.1834534 | 3.3165246 | 13 |
| 4 | 7.0636339 | 3.6822052 | 13 | 3.0011873 | 4.279804 | 13 | 6.8730869 | 3.0296104 | 13 |
| 7 | 8.3959052 | 3.6970511 | 13 | 2.4068579 | 4.0702307 | 13 | 6.2602932 | 2.6446528 | 13 |
| 9 | 9.6960336 | 4.190404 | 13 | 4.2797275 | 4.0123717 | 13 | 7.0100454 | 2.8612889 | 13 |
| 11 | 11.1210657 | 4.5962658 | 13 | 4.496152 | 4.1939636 | 13 | 7.668049 | 2.8675434 | 13 |
| 15 | 13.102226 | 5.2525782 | 13 | 7.9140611 | 5.2474548 | 13 | 9.9850326 | 3.0585043 | 13 |
| 16 | 14.407905 | 5.4190699 | 13 | 8.4805843 | 4.8019046 | 11 | 11.626924 | 3.7116911 | 12 |
| 18 | 13.254147 | 5.5683011 | 13 | 10.344788 | 4.3066984 | 11 | 10.378468 | 3.3180765 | 12 |
| 21 | 15.940184 | 3.4543543 | 11 | 9.2196447 | 3.951199 | 11 | 10.189654 | 3.0927191 | 12 |

| | mCCL19 + S16A | | | mCCL19 + SecSig only_S16A | | | SecSig only_S16A | | |
|---|---|---|---|---|---|---|---|---|---|
| Days | mean | SD | N | mean | SD | N | mean | SD | N |
| −14 | 0 | 0 | 13 | 0 | 0 | 13 | 0 | 0 | 13 |
| −13 | 0.3363469 | 2.849276 | 13 | −0.116557 | 2.6235486 | 13 | −0.309505 | 1.8179041 | 13 |
| −10 | 0.5399017 | 3.6302872 | 13 | 0.6659715 | 2.2319833 | 13 | −0.856511 | 2.7405685 | 13 |
| −7 | 2.046673 | 3.1052509 | 13 | 1.9990015 | 2.4132224 | 13 | 2.0901239 | 2.6036258 | 13 |
| −5 | 1.2998661 | 3.9683796 | 13 | 2.3140846 | 3.5211091 | 13 | 1.9946168 | 3.0938226 | 13 |
| −3 | 1.7950791 | 4.7893222 | 13 | 2.3299099 | 3.8583017 | 13 | 2.28857 | 3.1360442 | 13 |
| 0 | 4.2355916 | 4.8691098 | 13 | 3.5191472 | 4.8268418 | 13 | 4.7952941 | 4.1107589 | 13 |
| 1 | 5.5265159 | 5.4295635 | 13 | 4.4592385 | 4.6489957 | 13 | 5.3686055 | 4.6343427 | 13 |
| 4 | 6.9732772 | 5.7256578 | 13 | 5.0415309 | 4.9076359 | 13 | 5.6706553 | 4.77519 | 13 |
| 7 | 7.7884873 | 5.3121054 | 13 | 4.9024561 | 4.5869409 | 13 | 6.3273279 | 4.3344147 | 13 |
| 9 | 10.113595 | 6.0080605 | 13 | 7.5243626 | 4.3852818 | 13 | 8.2943677 | 5.1991386 | 13 |
| 11 | 11.198316 | 6.6055411 | 13 | 8.363441 | 4.4455726 | 13 | 8.6891917 | 4.9273341 | 13 |
| 15 | 13.045409 | 6.9021566 | 13 | 11.316722 | 3.9478518 | 13 | 9.7067576 | 5.8957347 | 13 |
| 16 | 13.006024 | 5.1353393 | 13 | 11.428477 | 5.0110762 | 12 | 11.375989 | 5.3506485 | 12 |
| 18 | 13.472109 | 5.0797797 | 13 | 11.816541 | 4.8421463 | 12 | 12.286157 | 5.4958607 | 12 |
| 21 | 14.609935 | 5.0919702 | 12 | 12.123724 | 6.9178058 | 10 | 12.881792 | 5.7666514 | 12 |

| | | |
|---|---|---|
| mCCL19 | S16A | Naïves |

| Days | mean | SD | N | mean | SD | N | mean | SD | N |
|---|---|---|---|---|---|---|---|---|---|
| −14 | 0 | 0 | 13 | 0 | 0 | 13 | 0 | 0 | 4 |
| −13 | 0.2011712 | 2.0668417 | 13 | 0.6493899 | 1.8358658 | 13 | 1.9587938 | 3.0031867 | 4 |
| −10 | 0.3715118 | 2.1207847 | 13 | −0.771033 | 2.46286 | 13 | −1.984108 | 0.9269933 | 4 |
| −7 | 3.2748973 | 2.60265 | 13 | 2.4137476 | 3.2404891 | 13 | 1.1103235 | 3.0020634 | 4 |
| −5 | 2.7416809 | 2.9657343 | 13 | 2.1622042 | 3.5767841 | 13 | 1.6642205 | 6.8774572 | 4 |
| −3 | 3.069653 | 3.2998084 | 13 | 1.4646322 | 3.8788568 | 13 | −0.146879 | 6.1310353 | 4 |
| 0 | 3.7361795 | 4.2160861 | 13 | 2.4367049 | 3.8790624 | 13 | −0.873845 | 5.1622466 | 4 |
| 1 | 4.7030953 | 4.9425986 | 13 | 1.3353305 | 3.155075 | 13 | 0.5438803 | 4.6703385 | 4 |
| 4 | 7.0207128 | 5.0954049 | 13 | 3.2412657 | 3.5901768 | 13 | 0.8475327 | 5.8467054 | 4 |
| 7 | 7.5854634 | 4.6983045 | 13 | 4.7469959 | 4.7736347 | 13 | −1.028012 | 4.4306542 | 4 |
| 9 | 9.8063817 | 6.215638 | 13 | 7.3217606 | 4.9660625 | 13 | 3.2754155 | 5.942024 | 4 |
| 11 | 10.064959 | 7.3266931 | 13 | 6.4435091 | 4.9761201 | 13 | 1.9369119 | 5.1442084 | 4 |
| 15 | 12.105985 | 6.1545446 | 13 | 8.3733404 | 4.2085153 | 13 | 4.9621835 | 6.5217102 | 4 |
| 16 | 13.613458 | 4.8249237 | 13 | 12.170933 | 4.5587239 | 13 | 10.136396 | 6.4251012 | 4 |
| 18 | 13.58406 | 6.044586 | 12 | 11.298329 | 4.7964645 | 13 | 8.4741938 | 7.0103076 | 4 |
| 21 | 14.558129 | 6.2365685 | 11 | 12.104161 | 4.5924463 | 11 | 6.3500841 | 5.749858 | 4 |

Conclusions

The Kolliphor delivered pUMVC4 plasmid vectors containing mCCL19, different multimerization units and the S16A neoepitopes resulted in CT26 anti-tumour effects from 50-100%. The groups receiving a DNA design with a multimerization unit that was of Ig origin or collagen performed the best. The neoepitope specific CD8+ T cell response at day 7 corresponded to the antitumor effects and the level of functional T cells at endpoint (as measured by IFNγ secretion) was comparable between groups receiving a DNA design containing neoepitopes. Furthermore, the DNA vaccines were well-tolerated by the mice.

### Example 5

### APC Targeting with Spike Protein from SARS-CoV-2

The aim of the study was to compare the performance of the APC(CLL19) targeting DNA plasmid using the spike receptor binding domain (RBD) delivered with Kolliphor with that of recombinant RBD adjuvanted with aluminium hydroxide. The goal was to determine the magnitude of the T cell response, the ability to induce a humoral antigen specific antibody response, and the ability of the generated antibodies to stop the virus from infecting cells in a micro-neutralization assay.

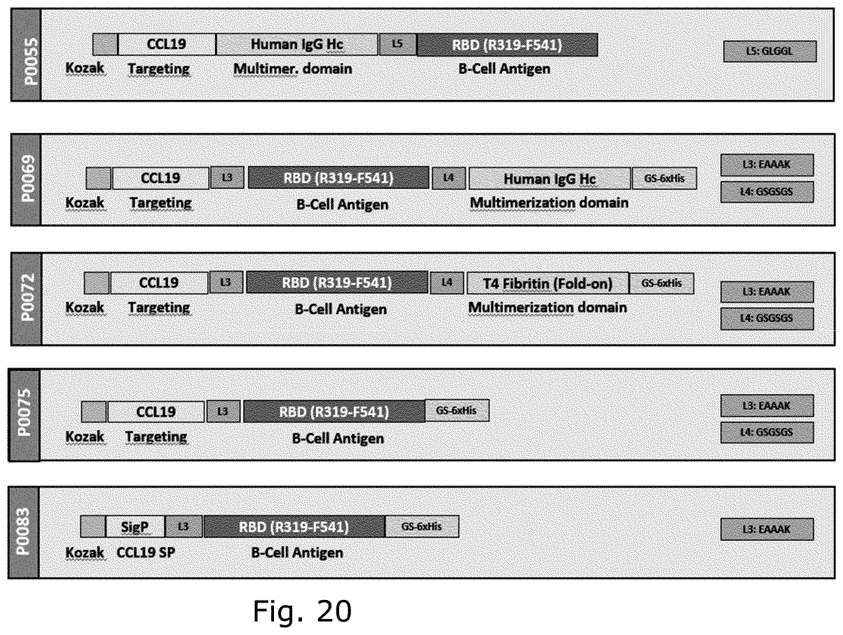

A number of vector designs were made, of which P0055 (see below) was evaluated in the current study. The selected RBD was taken from full length SARS-CoV-2 Spike protein (Acc ID: YP_009724390.1) by excising amino acids Arg319 to Phe541. The vector designs are shown schematically in FIG. 20.

Compared to the tested construct P0055, P0069 has been constructed with an aim to increase the expression level. Likewise, P0072 has been constructed with an aim to 1) increase expression levels, and 2) to form trimers instead of dimers of the expression product but remain to be tested.

P0075 and P0083 are controls, designed to investigate the effects of multimerization and CCL19 targeting respectively.

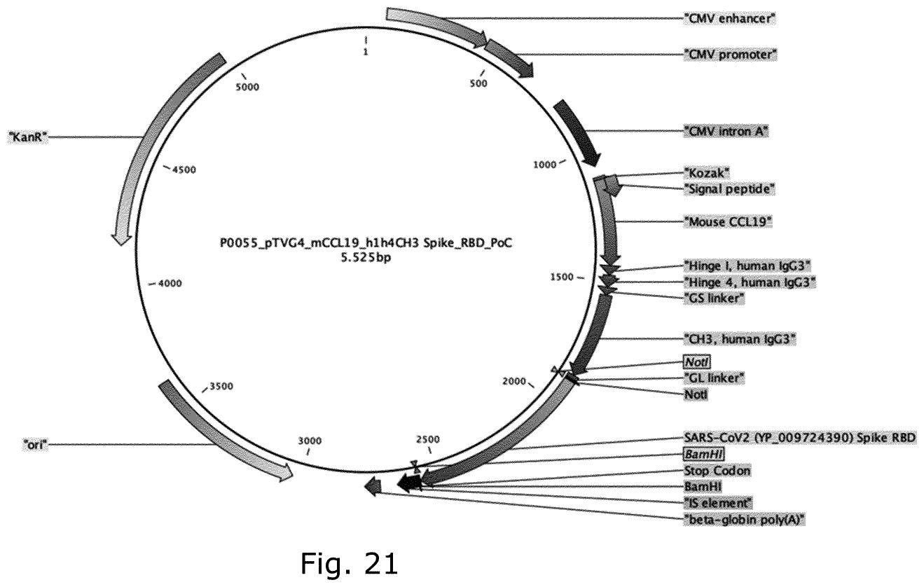

The expression vector P0055 is shown as a plasmid map in FIG. 21 and listed as SEQ ID NO 67. The RBD sequence encoded by the plasmid is provided as SEQ ID NO: 68.

Animal Study Details

Mice received immunizations with the test DNA plasmids on days days 0, 7, 14, 21, and 28. Each immunization consisted of intramuscular injection of 50 μl DNA plasmid in the left and right tibialis anterior, respectively. The dosage regimen for the recombinant RBD vaccines were 20 μg protein for the prime immunization, followed by two 20 μg boosting immunization at days 14 and 28. Each immunization consisted of subcutaneous injection of 100 μl RBD+aluminium hydroxide vaccine. Animals were sacrificed on day 35.

4 groups (1-4) of mice (5, 7, 7, and 5 individuals, respectively) were assigned to the following treatments:
Group 1: Plasmid P0053—pTGV4-CCL19 (Mock, i.e. DNA plasmid without spike protein), 25 μg.
Group 2: Plasmid P0055 (i.e. DNA plasmid pTGV4 with targeting unit CCL19 and RBD from spike protein), 25 μg.
Group 3. RBD protein in 2% Alhydrogel, 20 μg.
Group 4. Alhydrogel 2%.

Read-outs were body weight variation (based on 3 weekly measurements) and measurements based on blood sampling (day 19 and at day 35) and spleen sampling (at day 35). Measurement performed were determination of anti-RBD IgG in blood, splenic T-cell activation (IFN-γ release ELISPOT on spleen cells restimulated with peptides), and neutralization of SARS-CoV-2 in vitro.

Results

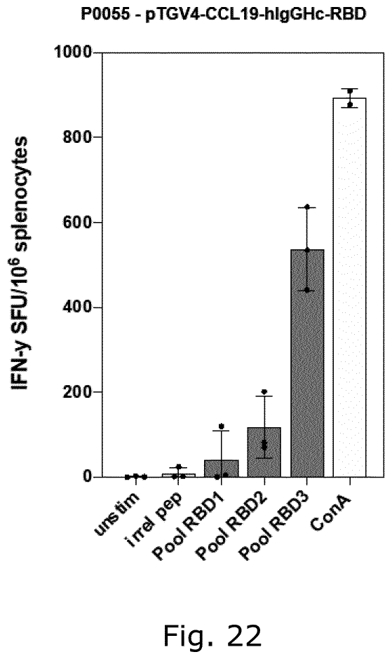

FIG. 22 shows a bar graph visualizing the IFN-γ release from the T-cells of mice from group 2. The T-cells were re-stimulated with either peptides or concanavalin A (conA, positive control for IFN-γ release). The two left-most bars show release from non-stimulated cells and cells stimulated with an irrelevant peptide. Pools 1, 2, and 3 are constituted by overlapping peptides from the RBD protein according to the following distribution:
Pool RBD1: CoV2_s-319, CoV2_s-325, CoV2_s-331, CoV2_s-337, CoV2_s-343, CoV2_s-349, CoV2_s-355, CoV2_s-361, CoV2_s-367, CoV2_s-373, CoV2_s-379, CoV2_s-385
Pool RBD2: CoV2_s-391, CoV2_s-397, CoV2_s-403, CoV2_s-409, CoV2_s-415, CoV2_s-421, CoV2_s-427, CoV2_s-439, CoV2_s-445, CoV2_s-451, CoV2_s-457
Pool RBD3: CoV2_s-463, CoV2_s-469, CoV2_s-475, CoV2_s-481, CoV2_s-487, CoV2_s-493, CoV2 s-499, CoV2 s-505, CoV2_s-511, CoV2 s-517, CoV2 s-523, CoV2_s-529

Cells stimulated with pools 2 and 3 exhibit a significantly higher release of IFN-γ than the two groups being non-stimulated or stimulated with irrelevant peptide.

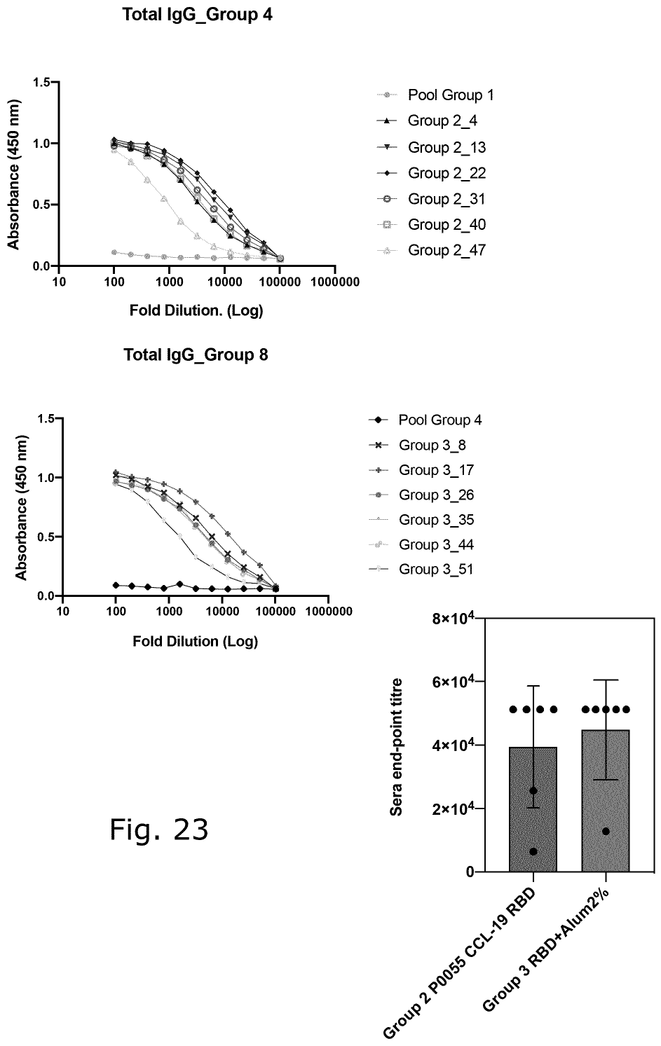

FIG. 23 shows ELISA measurements of total RBD Specific IgG in serum in mice from day 35 as pooled placebo sera for group 1 and group 4, while individual mice were assayed for group 2 and group 3. The resulting end-point titters are shown in FIG. 23 as a bar graph. Immunization with P0055 CCL-19 RBD shows successful extracellular expression of the RBD protein in the immunized mice with a resulting potent humoral response as antibody titers comparable to recombinant Spike RBD with alumhydroxid is achieved.

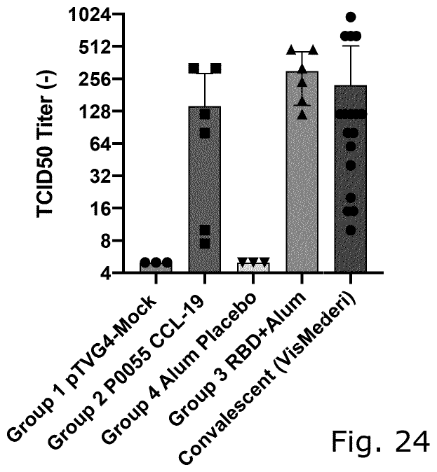

Finally, FIG. 24 shows a bar graphs visualizing the neutralizing effect of antibodies in the 4 tested groups and antibodies from convalescent patient sera. As is clear, the mice from group 2 produces antibodies that are comparable in neutralizing efficacy with those produced by group 3 and convalescent patients. This supports the application of APC targeting with the spike protein or fragments thereof.

Conclusions

The constructs of the present invention induce strong T cell response using a spike RBD design. Further, a humoral response can be induced with a Spike RBD design according to the present invention in same magnitude as with recombinant Spike RBD+2% Alum. Finally, the induced antibody titers show a neutralization efficiency comparable to convalescent patient sera.

Apc Targeting Units According to the Invention:

| Molecule | *Target cell type | *Other target cell type | SEQ ID |
|---|---|---|---|
| mCCL3 | Immature dendritic cell | Monocyte/macrophage, T cell (TH1 > TH2), | SEQ ID NO: 36 |
| | Receptors; CCR1 and CCR5 | NK cell, basophil, eosinophil, neutrophil, | |
| | | astrocyte, fibroblast, osteoclast | |
| mCCL4 | Immature dendritic cell | Monocyte/macrophage, T cell (TH1 > TH2), | SEQ ID NO: 37 |
| | Receptors; CCR5>> and CCR1 | NK cell, basophil, | |
| | | eosinophil, B cell | |
| mCCL5 | Immature dendritic cell | Monocyte/macrophage, T cell (memory T cell > | SEQ ID NO: 38 |
| | Receptors; CCR1, CCR3 and | T cell; TH1 > TH2), NK cell, basophil, | |
| | CCR5 | eosinophil | |
| mCCL19 | Mature dendritic cells | Naive T cells, B cells | SEQ ID NO: 39 |
| | Receptors; CCR7 | | |
| mXcl1 | CD8α + dendritic cells | T cells, natural killer cells, | SEQ ID NO: 40 |
| mCCL20 | Immature dendritic cell | T cells (memory T cells, TH17 cells), blood | SEQ ID NO: 41 |
| | Receptors; CCR6 | mononuclear cells, | |
| | | activated B cells, NKT cells, GALT | |
| | | development | |
| mCCL21 | Mature dendritic cell | Naive T cells, B cells, thymocytes, NK cells, | SEQ ID NO: 42 |
| | Receptors; CCR7 | | |
| GM-CSF | GM-CSF binds the receptor | Stimulates growth and differentiation | SEQ ID NO: 43 |
| | CD116, which is expressed | of myelomonocytic lineage cells, | |
| | on monocytes, neutrophils, | particularly dendritic cells. | |
| | eosinophils | | |
| anti-murine DEC- | Dendritic cells | DEC205 is a receptor found on dendritic cells | SEQ ID NO: 45 |
| 205 Fv | | and is a putative antigen uptake receptor | |
| anti-murine | Dendritic cells | Clec9 (CD370) is a receptor expressed on | SEQ ID NO: 46 |
| CLEC9 Fv | | dendritic cells, NK cells. Important for cross- | |
| | | priming of CD8 T cells for antiviral immunity | |
| CLEC9 ligand | As above | As above | SEQ ID NO: 47 |

SEQUENCES

SEQ ID NO: 1:
GSGGGA
SEQ ID NO: 2:
GSGGGAGSGGGA
SEQ ID NO: 3:
GSGGGAGSGGGAGSGGGA
SEQ ID NO: 4:
GSGGGAGSGGGAGSGGGAGSGGGA
SEQ ID NO: 5:
GENLYFQSGG
SEQ ID NO: 6:
KPEPKPAPAPKP
SEQ ID NO: 7:
AEAAAKEAAAKA
SEQ ID NO: 8:
SACYCELS
SEQ ID NO: 9:
SGGGSSGGGS
SEQ ID NO: 10:
GGGGSGGGGS
SEQ ID NO: 11:
SSGGGSSGGG
SEQ ID NO: 12:
GGSGGGGSGG
SEQ ID NO: 13:
GSGSGSGSGS
SEQ ID NO: 14:
ggGGTCAACGTTGAgggggg
SEQ ID NO: 15:
ggGGGACGATCGTCgggggg
SEQ ID NO: 16:
gggGACGACGTCGTGgggggg
SEQ ID NO: 17:
tccatgacgttcctgatgct
SEQ ID NO: 18:
tccatgacgttcctgacgtt
SEQ ID NO: 19:
tcgtcgttttgtcgttttgtcgtt
SEQ ID NO: 20:
tcgtcgttgtcgttttgtcgtt
SEQ ID NO: 21:
tcgacgttcgtcgttcgtcgttc
SEQ ID NO: 22:
tcgcgacgttcgcccgacgttcggta
SEQ ID NO: 23:
tcgtcgttttcggcgcgcgccg
SEQ ID NO: 24:
tcgtcgtcgttcgaacgacgttgat
SEQ ID NO: 25:
tcgcgaacgttcgccgcgttcgaacgcgg
SEQ ID NO: 26:
agatctaacgacaaaacgacaaaacgacaaggcgccagatctggcgtttcgttttgtcgttttgtcgttagatct
SEQ ID NO: 27:
GGTGCATCGA TGCAGGGGGG
SEQ ID NO: 28:
GGTGCATCGA TGCAGGGGGG TATATATATA TTGAGGACAG GTTAAGCTCC CCCCAGCTTA ACCTGTCCTT
CAATATATA TATA
SEQ ID NO: 29: vector backbone; pUMVC4a from aldevron
tggccattgcatacgttgtatccatatcataatatgtacatttatattggctcatgtccaacattaccgccatgttgacattg
attattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatcatatgcc
aagtacgccccctattgacgtcaatgacggtaaatggcccgcctggcattatgcccagtacatgaccttatgggactttccta
cttggcagtacatctacgtattagtcatcgctattaccatggtgatgcggttttggcagtacatcaatgggcgtggatagcgg
tttgactcacggggatttccaagtctccaccccattgacgtcaatgggagtttgttttggcaccaaaatcaacgggactttcc
aaaatgtcgtaacaactccgccccattgacgcaaatgggcggtaggcgtgtacggtgggaggtctatataagcagagctcgtt
tagtgaaccgtcagatcgcctggagacgccatccacgctgttttgacctccatagaagacaccgggaccgatccagcctccgc
ggccgggaacggtgcattggaacgcggattccccgtgccaagagtgacgtaagtaccgcctatagagtctataggcccacccc
cttggcttcttatgcatgctatactgtttttggcttggggtctatacacccccgcttcctcatgttataggtgatggtatagc
ttagcctataggtgtgggttattgaccattattgaccactccaacggtggagggcagtgtagtctgagcagtactcgttgctg
cggtatcgataagcttgatatcgaattcacgtgggcccggtaccgtatactctagagcggccgcggatccagatctttttccc
tcgccaaaaattatggggacatcatgaagccccttgagcatctgacttctggctaataaaggaaatttatttcattgcaatag
tgtgttggaattttttgtgtctctcactcggaaggacatatgggagggcaaatcatttaaaacatcagaatcagtatttggtt
tagagtttggcaacatatgccattcttccgcttcctcgctcactgactcgctgcgctcggtcgttcggctgcggcgagcggta
tcagctcactcaaaggcggtaatacggttatccacagaatcaggggataacgcaggaaagaacatgtgagcaaaaggccagca
aaaggccaggaaccgtaaaaaggccgcgttgctggcgtttttccataggctccgcccccctgacgagcatcacaaaaatcgac
gctcaagtcagaggtggcgaaacccgacaggactataaagataccaggcgtttccccctggaagctccctcgtgcgctctcct
gttccgaccctgccgcttaccggatacctgtccgcctttctcccttcgggaagcgtggcgctttctcaatgctcacgctgtag
gtatctcagttcggtgtaggtcgttcgctccaagctgggctgtgtgcacgaaccccccgttcagcccgaccgctgcgccttat
ccggtaactatcgtcttgagtccaacccggtaagacacgacttatcgccactggcagcagccactggtaacaggattagcaga
gcgaggtatgtaggcggtgctacagagttcttgaagtggtggcctaactacggctacactagaaggacagtatttggtatctg
cgctctgctgaagccagttaccttcggaaaaagagttggtagctcttgatccggcaaacaaaccaccgctggtagcggtggtt
tttttgtttgcaagcagcagattacgcgcagaaaaaaaggatctcaagaagatcctttgatcttttctacggggtctgacgct
cagtggaacgaaaactcacgttaagggattttggtcatgagattatcaaaaaggatcttcacctagatccttttaaattaaaa
atgaagttttaaatcaatctaaagtatatatgagtaaacttggtctgacagttaccaatgcttaatcagtgaggcacctatct
cagcgatctgtctatttcgttcatccatagttgcctgactccggggggggggggcgctgaggtctgcctcgtgaagaaggtgt
tgctgactcataccagggcaacgttgttgccattgctacaggcatcgtggtgtcacgctcgtcgtttggtatggcttcattca
gctccggttcccaacgatcaaggcgagttacatgatcccccatgttgtgcaaaaaagcggttagctccttcggtcctccgatc
gttgtcagaagtaagttggccgcagtgttatcactcatggttatggcagcactgcataattctcttactgtcatgccatccgt
aagatgcttttctgtgactggtgagtactcaaccaagtcattctgagaatagtgtatgcggcgaccgagttgctcttgcccgg
cgtcaatacgggataataccgcgccacatagcagaactttaaaagtgctcatcattggaaaacgttcttcggggcgaaaactc
tcaaggatcttaccgctgttgagatccagttcgatgtaacccactcgtgcacctgaatcgccccatcatccagccagaaagtg
agggagccacggttgatgagagctttgttgtaggtggaccagttggtgattttgaacttttgctttgccacggaacggtctgc
gttgtcgggaagatgcgtgatctgatccttcaactcagcaaaagttcgatttattcaacaaagccgccgtcccgtcaagtcag
cgtaatgctctgccagtgttacaaccaattaaccaattctgattagaaaaactcatcgagcatcaaatgaaactgcaatttat
tcatatcaggattatcaataccatatttttgaaaaagccgtttctgtaatgaaggagaaaactcaccgaggcagttccatagg
atggcaagatcctggtatcggtctgcgattccgactcgtccaacatcaatacaacctattaatttcccctcgtcaaaaataag
gttatcaagtgagaaatcaccatgagtgacgactgaatccggtgagaatggcaaaagcttatgcatttctttccagacttgtt
caacaggccagccattacgctcgtcatcaaaatcactcgcatcaaccaaaccgttattcattcgtgattgcgcctgagcgaga
cgaaatacgcgatcgctgttaaaaggacaattacaaacaggaatcgaatgcaaccggcgcaggaacactgccagcgcatcaac
aatattttcacctgaatcaggatattcttctaatacctggaatgctgttttcccggggatcgcagtggtgagtaaccatgcat
catcaggagtacggataaaatgcttgatggtcggaagaggcataaattccgtcagccagtttagtctgaccatctcatctgta
acatcattggcaacgctacctttgccatgtttcagaaacaactctggcgcatcgggcttcccatacaatcgatagattgtcgc
acctgattgcccgacattatcgcgagcccatttatacccatataaatcagcatccatgttggaatttaatcgcggcctcgagc
aagacgtttcccgttgaatatggctcataacaccccttgtattactgtttatgtaagcagacagttttattgttcatgatgat
atatttttatcttgtgcaatgtaacatcagagattttgagacacaacgtggctttccccccccccccattattgaagcattta
tcagggttattgtctcatgagcggatacatatttgaatgtatttagaaaaataaacaaataggggttccgcgcacatttcccc
gaaaagtgccacctgacgtctaagaaaccattattatcatgacattaacctataaaaataggcgtatcacgaggccctttcgt
cctcgcgcgtttcggtgatgacggtgaaaacctctgacacatgcagctcccggagacggtcacagcttgtctgtaagcggatg
ccgggagcagacaagcccgtcagggcgcgtcagcgggtgttggcgggtgtcggggctggcttaactatgcggcatcagagcag
attgtactgagagtgcaccatatgcggtgtgaaataccgcacagatgcgtaaggagaaaataccgcatcagattggctat
SEQ ID NO: 30: enhancer, start Kozak sequence without ATG
CGCCGCCACC
SEQ ID NO: 31: dimer Hinge 1, human IgG3
GAGCTCAAAACCCCACTTGGTGACACAACTCACACA
SEQ ID NO: 32: dimer Hinge 4, human IgG3
GAGCCCAAATCTTGTGACACACCTCCCCCGTGCCCAAGGTGCCCA
SEQ ID NO: 33: dimer Gly-ser linker
GGCGGTGGAAGCAGCGGAGGTGGAAGTGGA
SEQ ID NO: 34: dimer CH3, human IgG3
GGACAGCCCCGAGAACCACAGGTGTACACCCTGCCCCCATCCCGGGAGGAGATGACCAAGAACCAGGTCAGCCTGACCTGCCT
GGTCAAAGGCTTCTACCCCAGCGACATCGCCGTGGAGTGGGAGAGCAGCGGGCAGCCGGAGAACAACTACAACACCACGCCTC
CCATGCTGGACTCCGACGGCTCCTTCTTCCTCTACAGCAAGCTCACCGTGGACAAGAGCAGGTGGCAGCAGGGGAACATCTTC
TCATGCTCCGTGATGCATGAGGCTCTGCACAACCGCTTCACGCAGAAGAGCCTCTCCCTGTCTCCGGGTAAA
SEQ ID NO: 35: dimer Gly-Leu linker
GGCCTCGGTGGCCTG
SEQ ID NO: 36: APC targeting mCCL3
ATGAAGGTCTCCACCACTGCCCTTGCTGTTCTTCTCTGTACCATGACACTCTGCAACCAAGTCTTCTCAGCGCCATATGGAGC
TGACACCCCGACTGCCTGCTGCTTCTCCTACAGCCGGAAGATTCCACGCCAATTCATCGTTGACTATTTTGAAACCAGCAGCC
TTTGCTCCCAGCCAGGTGTCATTTTCCTGACTAAGAGAAACCGGCAGATCTGCGCTGACTCCAAAGAGACCTGGGTCCAAGAA
TACATCACTGACCTGGAACTGAATGCC
SEQ ID NO: 37: APC targeting mCCL4
ATGAAGCTCTGCGTGTCTGCCCTCTCTCTCCTCTTGCTCGTGGCTGCCTTCTGTGCTCCAGGGTTCTCAGCACCAATGGGCTC
TGACCCTCCCACTTCCTGCTGTTTCTCTTACACCTCCCGGCAGCTTCACAGAAGCTTTGTGATGGATTACTATGAGACCAGCA
GTCTTTGCTCCAAGCCAGCTGTGGTATTCCTGACCAAAAGAGGCAGACAGATCTGTGCTAACCCCAGTGAGCCCTGGGTCACT
GAGTACATGAGTGACTTGGAG
SEQ ID NO: 38: APC targeting mCCL5
ATGAAGATCTCTGCAGCTGCCCTCACCATCATCCTCACTGCAGCCGCCCTCTGCACCCCCGCACCTGCCTCACCATATGGCTC
GGACACCACTCCCTGCTGCTTTGCCTACCTCTCCCTCGCGCTGCCTCGTGCCCACGTCAAGGAGTATTTCTACACCAGCAGCA
AGTGCTCCAATCTTGCAGTCGTGTTTGTCACTCGAAGGAACCGCCAAGTGTGTGCCAACCCAGAGAAGAAGTGGGTTCAAGAA
TACATCAACTATTTGGAGATGAGC
SEQ ID NO: 39 APC targeting mCCL19
ATGGCCCCCCGTGTGACCCCACTCCTGGCCTTCAGCCTGCTGGTTCTCTGGACCTTCCCAGCCCCAACTCTGGGGGGTGCTAA
TGATGCGGAAGACTGCTGCCTGTCTGTGACCCAGCGCCCCATCCCTGGGAACATCGTGAAAGCCTTCCGCTACCTTCTTAATG
AAGATGGCTGCAGGGTGCCTGCTGTTGTGTTCACCACACTAAGGGGCTATCAGCTCTGTGCACCTCCtGACCAGCCCTGGGTG
GATCGCATCATCCGAAGACTGAAGAAGTCTTCTGCCAAGAACAAAGGCAACAGCACCAGAAGGAGCCCTGTGTCT
SEQ ID NO: 40: APC targeting mxcl1
ATGAGACTTCTCCTCCTGACTTTCCTGGGAGTCTGCTGCCTCACCCCATGGGTTGTGGAAGGTGTGGGGACTGAAGTCCTAGA
AGAGAGTAGCTGTGTGAACTTACAAACCCAGCGGCTGCCAGTTCAAAAAATCAAGACCTATATCATCTGGGAGGGGGCCATGA
GAGCTGTAATTTTTGTCACCAAACGAGGACTAAAAATTTGTGCTGATCCAGAAGCCAAATGGGTGAAAGCAGCGATCAAGACT
GTGGATGGCAGGGCCAGTACCAGAAAGAACATGGCTGAAACTGTTCCCACAGGAGCCCAGAGGTCCACCAGCACAGCGATAAC
CCTGACTGGG
SEQ ID NO: 41: APC targeting mCCL20
ATGGCCTGCGGTGGCAAGCGTCTGCTCTTCCTTGCTTTGGCATGGGTACTGCTGGCTCACCTCTGCAGCCAGGCAGAAGCAGC
AAGCAACTACGACTGTTGCCTCTCGTACATACAGACGCCTCTTCCTTCCAGAGCTATTGTGGGTTTCACAAGACAGATGGCCG
ATGAAGCTTGTGACATTAATGCTATCATCTTTCACACGAAGAAAAGAAAATCTGTGTGCGCTGATCCAAAGCAGAACTGGGTG
AAAAGGGCTGTGAACCTCCTCAGCCTAAGAGTCAAGAAGATG
SEQ ID NO: 42: APC targeting mCCL21
ATGGCTCAGATGATGACTCTGAGCCTCCTTAGCCTGGTCCTGGCTCTCTGCATCCCCTGGACCCAAGGCAGTGATGGAGGGGG
TCAGGACTGCTGCCTTAAGTACAGCCAGAAGAAAATTCCCTACAGTATTGTCCGAGGCTATAGGAAGCAAGAACCAAGTTTAG
GCTGTCCCATCCCGGCAATCCTGTTCTCACCCCGGAAGCACTCTAAGCCTGAGCTATGTGCAAACCCTGAGGAAGGCTGGGTG
CAGAACCTGATGCGCCGCCTGGACCAGCCTCCAGCCCCAGGGAAACAAAGCCCCGGCTGCAGGAAGAACCGGGGAACCTCTAA
GTCTGGAAAGAAAGGAAAGGGCTCCAAGGGCTGCAAGAGAACTGAACAGACACAGCCCTCAAGAGGA
SEQ ID NO: 43: APC targeting GM-CSF
ATGTGGCTGCAGAATTTACTTTTCCTGGGCATTGTGGTCTACAGCCTCTCAGCACCCACCCGCTCACCCATCACTGTCACCCG
GCCTTGGAAGCATGTAGAGGCCATCAAAGAAGCCCTGAACCTCCTGGATGACATGCCTGTCACGTTGAATGAAGAGGTAGAAG
TCGTCTCTAACGAGTTCTCCTTCAAGAAGCTAACATGTGTGCAGACCCGCCTGAAGATATTCGAGCAGGGTCTACGGGGCAAT
TTCACCAAACTCAAGGGCGCCTTGAACATGACAGCCAGCTACTACCAGACATACTGCCCCCCAACTCCGGAAACGGACTGTGA
AACACAAGTTACCACCTATGCGGATTTCATAGACAGCCTTAAAACCTTTCTGACTGATATCCCCTTTGAATGCAAAAAACCAG
GCCAAAAA
SEQ ID NO: 44: secretion signal on APC t Secretion signal, mouse serum albumin
ATGAAATGGGTGACCTTTCTGCTGCTGCTGTTTGTGAGCGGCAGCGCGTTTAGCG
SEQ ID NO: 45: APC targeting anti-murine DEC-205 Fv
CGGCCCAGCCGGCCATGGCGGACTACAAACAGGCTGTTGTGACTCAGGAATCAGCACTCACCACATCACCTGGTGAAACAGTC
ACACTCACTTGTCGCTCAAGTACTGGGGCTGTTACAATTAGTAACTATGCCAACTGGGTCCAAGAAAAACCAGATCATTTATT
CACTGGTCTAATAGGTGGTACCAACAACCGAGCTCCAGGTGTTCCTGCCAGATTCTCAGGCTCCCTGATTGGAGACAAGGCTG
CCCTCACCATCACAGGGGCACAGACTGAGGATGAGGCAATCTATTTCTGTGCTCTATGGTACAACAACCAGTTTATTTTCGGC
AGTGGAACCAAGGTCACTGTCCTAGGTGGTGGTGGTGGTTCTGGTGGTGGTGGATCCGGCGGCGGCGGCTCTGGCGGCGGCGG
CTCTGAGGTCCAGCTGCAACAGTCTGGACCTGTGCTGGTGAAGCCTGGGGCTTCAGTGAAGATGTCCTGTAAGGCTTCTGGAA
ACACATTCACTGACTCCTTTATGCACTGGATGAAACAGAGCCATGGAAAGAGTCTTGAGTGGATTGGAATTATTAATCCTTAT
AACGGCGGTACTAGCTACAACCAGAAATTCAAGGGCAAGGCCACATTGACTGTTGACAAGTCCTCCAGCACAGCCTACATGGA
GCTCAACAGCCTGACATCTGAGGACTCTGCAGTCTATTACTGTGCAAGAAACGGGGTGCGGTACTACTTTGACTACTGGGGCC
AAGGCACCACTCTCACAGTCTCCTCGGCCTCGGGGGCC
SEQ ID NO: 46: APC targeting anti-murine CLEC9 Fv
GCGGCCCAGCCGGCCATGGCGGACTACAAACAGGCTGTTGTGACTCAGGAATCAGCACTCACCACATCACCTGGTGAAACAGT
CACACTCACTTGTCGCTCAAGTAAAAGCAGCCAGAGCGTGCTGTATGATGAAAACAAAAAAAACTATCTGGCCAACTGGGTCC
AAGAAAAACCAGATCATTTATTCACTGGTCTAATAGGTTGGGCGAGCACCGGCGAAAGCAACCGAGCTCCAGGTGTTCCTGCC
AGATTCTCAGGCTCCCTGATTGGAGACAAGGCTGCCCTCACCATCACAGGGGCACAGACTGAGGATGAGGCAATCTATTTCTG
TTATTATGATTTTCCGCCGTTCGGCAGTGGAACCAAGGTCACTGTCCTAGGTGGTGGTGGTGGTTCTGGTGGTGGTGGATCCG
GCGGCGGCGGCTCTGGCGGCGGCGGCTCTGAGGTCCAGCTGCAACAGTCTGGACCTGTGCTGGTGAAGCCTGGGGCTTCAGTG
AAGATGTCCTGTAAGGCTTCTAACGCGGCGATTTATATGCACTGGATGAAACAGAGCCATGGAAAGAGTCTTGAGTGGATTGG
AATTCGCATTCGCACCCGCCCGAGCAAATATGCGACCGATTATGCGGATAGCGTGCGCGGCAGCTACAACCAGAAATTCAAGG
GCAAGGCCACATTGACTGTTGACAAGTCCTCCAGCACAGCCTACATGGAGCTCAACAGCCTGACATCTGAGGACTCTGCAGTC
TATTACTGTCGCGCGACCGAAGATGTGCCGTTTTATTACTGGGGCCAAGGCACCACTCTCACAGTCTCCTCGGCCTCGGGGGC
C
SEQ ID NO: 47: APC targeting CLEC9 ligand TGGCCCAGGTTCCACAGCAGCGTGTTCCACACCCAC
SEQ ID NO: 48: APC targeting mFLT3L
ATGACACCTGACTGTTACTTCAGCCACAGTCCCATCTCCTCCAACTTCAAAGTGAAGTTTAGAGAGTTGACTGACCACCTGCT
TAAAGATTACCCAGTCACTGTGGCCGTCAATCTTCAGGACGAGAAGCACTGCAAGGCCTTGTGGAGCCTCTTCCTAGCCCAGC
GCTGGATAGAGCAACTGAAGACTGTGGCAGGGTCTAAGATGCAAACGCTTCTGGAGGACGTCAACACCGAGATACATTTTGTC
ACCTCATGTACCTTCCAGCCCCTACCAGAATGTCTGCGATTCGTCCAGACCAACATCTCCCACCTCCTGAAGGACACCTGCAC
ACAGCTGCTTGCTCTGAAGCCCTGTATCGGGAAGGCCTGCCAGAATTTCTCTCGGTGCCTGGAGGTGCAGTGCCAGCCGGACT
CCTCCACCCTGCTGCCCCCAAGGAGTCCCATAGCCCTAGAAGCCACGGAGCTCCCAGAGCCTCGGCCCAGGCAG
SEQ ID NO: 49: dimer, dHLX protein
GGAGAACTGGAGGAATTACTTAAACATCTCAAGGAGTTGCTCAAAGGCCCTAGGAAGGGAGAACTGGAGGAACTCCTCAAACA
TCTCAAGGAGTTACTAAAGGGA
SEQ ID NO: 50: dimer, GS-linker
GGCAGCGGCAGCGGCAGCGGCAGCGGCAGC
SEQ ID NO: 51: dimer, hMHD2 (human IgM)
GCCGAACTCCCGCCCAAGGTGTCCGTGTTCGTCCCTCCCCGCGATGGGTTCTTCGGCAATCCACGAAAATCCAAACTGATTTG
TCAGGCCACCGGCTTCTCCCCCCGACAGATCCAGGTGAGTTGGCTACGAGAGGGTAAACAGGTGGGGAGCGGAGTGACCACTG
ACCAGGTGCAGGCCGAGGCCAAGGAAAGCGGACCCACAACATACAAAGTGACAAGCACTCTGACGATTAAGGAGTCAGACTGG
CTCGGCCAATCCATGTTTACATGCCGGGTTGATCACAGAGGGTTGACCTTCCAACAGAACGCATCCAGTATGTGCGTTCCAGA
T
SEQ ID NO: 52: Trimer Collagen trimerisation domain
GCTGGGCAGGTGAGGATCTGGGCCACATACCAGACCATGCTGGACAAGATCCGGGAGGTGCCGGAGGGCTGGCTCATCTTTGT
GGCCGAGAGGGAAGAGCTCTATGTACGCGTTAGAAATGGCTTCCGGAAGGTGCTGCTGGAGGCCCGGACAGCCCTCCCGAGAG
GCACGGGCAATGAG
SEQ ID NO: 53: Tetramer p53 synthetic protein *
AAGCCTCTGGACGGAGAGTATTTCACTCTCCAGATCCGGGGCCCCGAAAGGTTCGAAATGTTCCGGGAGCTTAACGAGGCCTT
GGAGCTGAAAGACGCACAGGCCGGAAAGGAACCG
SEQ ID NO: 54:
QIETQQRKFKASRASILSEMKMLKEKR
SEQ ID NO: 55:
VILPQAPSGPSYATYLQPAQAQMLTPP
SEQ ID NO: 56:
DTLSAMSNPRAMQVLLQIQQGLQTLAT
SEQ ID NO: 57:
DGQLELLAQGALDNALSSMGALHALPR
SEQ ID NO: 58:
RLHVVKLLASALSTNAAALTqELLVLD
SEQ ID NO: 59: dimer Hinge 1, human IgG3 Protein sequence:
ELKTPLGDTTHT
SEQ ID NO: 60: dimer Hinge 4, human IgG3 PROTEIN SEQUENCE:
EPKSCDTPPPCPRCP
SEQ ID NO: 61 C22 NEOEPITOPE AMINO ACID SEQUENCE:
QIETQQRKFKASRASILSEMKMLKEKR
SEQ ID NO: 62 C23 neoepitope amino acid sequence:
VILPQAPSGPSYATYLQPAQAQMLTPP
SEQ ID NO: 63 C25 neoepitope amino acid sequence:
DTLSAMSNPRAMQVLLQIQQGLQTLAT
SEQ ID NO: 64 C30 neoepitope amino acid sequence:
DGQLELLAQGALDNALSSMGALHALPR
SEQ ID NO: 65 C38 neoepitope amino acid sequence:
RLHVVKLLASALSTNAAALTQELLVLD
SEQ ID NO: 66: Alternative GS-linker:
GGGSS
SEQ ID NO: 67 >P0055_pTVG4_mCCL19_h1h4CH3_Spike_RBD:
TGGCCATTGCATACGTTGTATCCATATCATAATATGTACATTTATATTGGCTCATGTCCAACATTACCGCCATGTTGACATTG
ATTATTGACTAGTTATTAATAGTAATCAATTACGGGGTCATTAGTTCATAGCCCATATATGGAGTTCCGCGTTACATAACTTA
CGGTAAATGGCCCGCCTGGCTGACCGCCCAACGACCCCCGCCCATTGACGTCAATAATGACGTATGTTCCCATAGTAACGCCA
ATAGGGACTTTCCATTGACGTCAATGGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGACCTTATGGGACTTTCCTA
CTTGGCAGTACATCTACGTATTAGTCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTGGCACCAAAATCAACGGGACTTTCC
AAAATGTCGTAACAACTCCGCCCCATTGACGCAAATGGGCGGTAGGCGTGTACGGTGGGAGGTCTATATAAGCAGAGCTCGTT
TAGTGAACCGTCAGATCGCCTGGAGACGCCATCCACGCTGTTTTGACCTCCATAGAAGACACCGGGACCGATCCAGCCTCCGC
GGCCGGGAACGGTGCATTGGAACGCGGATTCCCCGTGCCAAGAGTGACGTAAGTACCGCCTATAGACTCTATAGGCACACCCC
TTTGGCTCTTATGCATGCTATACTGTTTTTGGCTTGGGGCCTATACACCCCCGCTTCCTTATGCTATAGGTGATGGTATAGCT
TAGCCTATAGGTGTGGGTTATTGACCATTATTGACCACTCCAACGGTGGAGGGCAGTGTAGTCTGAGCAGTACTCGTTGCTGC
CGCGCGCGCCACCAGACATAATAGCTGACAGACTAACAGACTGTTCCTTTCCATGGGTCTTTTCTGCAGTCACCGTCGTCGAC
GGTATCGATAAGCTTGATATCGAATTCCGCCGCCACCATGGCCCCCCGTGTGACCCCACTCCTGGCCTTCAGCCTGCTGGTTC
TCTGGACCTTCCCAGCCCCAACTCTGGGGGGTGCTAATGATGCGGAAGACTGCTGCCTGTCTGTGACCCAGCGCCCCATCCCT
GGGAACATCGTGAAAGCCTTCCGCTACCTTCTTAATGAAGATGGCTGCAGGGTGCCTGCTGTTGTGTTCACCACACTAAGGGG
CTATCAGCTCTGTGCACCTCCTGACCAGCCCTGGGTGGATCGCATCATCCGAAGACTGAAGAAGTCTTCTGCCAAGAACAAAG
GCAACAGCACCAGAAGGAGCCCTGTGTCTGAGCTCAAAACCCCACTTGGTGACACAACTCACACAGAGCCCAAATCTTGTGAC
ACACCTCCCCCGTGCCCAAGGTGCCCAGGCGGTGGAAGCAGCGGAGGTGGAAGTGGAGGACAGCCCCGAGAACCACAGGTGTA
CACCCTGCCCCCATCCCGGGAGGAGATGACCAAGAACCAGGTCAGCCTGACCTGCCTGGTCAAAGGCTTCTACCCCAGCGACA
TCGCCGTGGAGTGGGAGAGCAGCGGGCAGCCGGAGAACAACTACAACACCACGCCTCCCATGCTGGACTCCGACGGCTCCTTC
TTCCTCTACAGCAAGCTCACCGTGGACAAGAGCAGGTGGCAGCAGGGGAACATCTTCTCATGCTCCGTGATGCATGAGGCTCT
GCACAACCGCTTCACGCAGAAGAGCCTCTCCCTGTCTCCGGGTAAAGGCCTCGGTGGCCTGGCGGCCGCGAGGGTGCAGCCGA
CTGAGAGTATCGTTCGTTTCCCTAACATTACGAACCTATGTCCGTTTGGAGAGGTATTTAATGCTACCAGATTCGCATCAGTC
TATGCTTGGAACAGGAAACGGATATCAAACTGCGTGGCCGACTATAGCGTTCTCTACAACAGTGCCTCTTTTAGCACTTTCAA
GTGCTACGGAGTCTCCCCAACCAAGCTAAACGACCTGTGCTTCACAAACGTCTACGCTGACTCCTTTGTCATCAGAGGCGATG
AGGTGCGGCAAATCGCACCTGGACAAACTGGAAAAATCGCCGACTATAACTATAAGCTGCCCGATGACTTTACTGGATGCGTG
ATCGCTTGGAATTCAAACAATCTCGACAGTAAGGTAGGAGGCAATTACAACTACCTTTATCGGCTCTTCCGAAAATCAAACCT
GAAACCCTTTGAGCGTGATATCTCGACTGAAATTTACCAAGCTGGCAGCACTCCTTGCAACGGAGTGGAGGGCTTCAATTGTT
ACTTTCCTCTGCAGAGCTACGGATTCCAACCTACAAACGGGGTAGGCTACCAACCCTACCGAGTGGTGGTGCTGAGCTTTGAG
CTGCTCCATGCTCCAGCAACTGTATGCGGCCCTAAGAAGTCAACTAACCTGGTTAAGAATAAATGCGTCAACTTTTAGGGATC
CAGATCTAACGACAAAACGACAAAACGACAAGGCGCCAGATCTGGCGTTTCGTTTTGTCGTTTTGTCGTTAGATCTTTTTCCC
TCTGCCAAAAATTATGGGGACATCATGAAGCCCCTTGAGCATCTGACTTCTGGCTAATAAAGGAAATTTATTTTCATTGCAAT
AGTGTGTTGGAATTTTTTGTGTCTCTCACTCGGAAGGACATATGGGAGGGCAAATCATTTAAAACATCAGAATGAGTATTTGG
TTTAGAGTTTGGCAACATATGCCCATTCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCTTTGATCTTTTCTACGGGGTCTGAC
GCTCAGTGGAACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTGAGGCACCTA
TCTCAGCGATCTGTCTATTTCGTTCATCCATAGTTGCCTGACTCGGGGGGGGGGGGCGCTGAGGTCTGCCTCGTGAAGAAGGT
GTTGCTGACTCATACCAGGCCTGAATCGCCCCATCATCCAGCCAGAAAGTGAGGGAGCCACGGTTGATGAGAGCTTTGTTGTA
GGTGGACCAGTTGGTGATTTTGAACTTTTGCTTTGCCACGGAACGGTCTGCGTTGTCGGGAAGATGCGTGATCTGATCCTTCA
ACTCAGCAAAAGTTCGATTTATTCAACAAAGCCGCCGTCCCGTCAAGTCAGCGTAATGCTCTGCCAGTGTTACAACCAATTAA
CCAATTCTGATTAGAAAAACTCATCGAGCATCAAATGAAACTGCAATTTATTCATATCAGGATTATCAATACCATATTTTTGA
AAAAGCCGTTTCTGTAATGAAGGAGAAAACTCACCGAGGCAGTTCCATAGGATGGCAAGATCCTGGTATCGGTCTGCGATTCC
GACTCGTCCAACATCAATACAACCTATTAATTTCCCCTCGTCAAAAATAAGGTTATCAAGTGAGAAATCACCATGAGTGACGA
CTGAATCCGGTGAGAATGGCAAAAGCTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAATACGCGATCGCTGTTAAAAGGACAATT
ACAAACAGGAATCGAATGCAACCGGCGCAGGAACACTGCCAGCGCATCAACAATATTTTCACCTGAATCAGGATATTCTTCTA
ATACCTGGAATGCTGTTTTCCCGGGGATCGCAGTGGTGAGTAACCATGCATCATCAGGAGTACGGATAAAATGCTTGATGGTC
GGAAGAGGCATAAATTCCGTCAGCCAGTTTAGTCTGACCATCTCATCTGTAACATCATTGGCAACGCTACCTTTGCCATGTTT
CAGAAACAACTCTGGCGCATCGGGCTTCCCATACAATCGATAGATTGTCGCACCTGATTGCCCGACATTATCGCGAGCCCATT
TATACCCATATAAATCAGCATCCATGTTGGAATTTAATCGCGGCCTCGAGCAAGACGTTTCCCGTTGAATATGGCTCATAACA
CCCCTTGTATTACTGTTTATGTAAGCAGACAGTTTTATTGTTCATGATGATATATTTTTATCTTGTGCAATGTAACATCAGAG
ATTTTGAGACACAACGTGGCTTTCCCCCCCCCCCCATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCACCTGACGTCTAAGAAACCATT
ATTATCATGACATTAACCTATAAAAATAGGCGTATCACGAGGCCCTTTCGTCTCGCGCGTTTCGGTGATGACGGTGAAAACCT
CTGACACATGCAGCTCCCGGAGACGGTCACAGCTTGTCTGTAAGCGGATGCCGGGAGCAGACAAGCCCGTCAGGGCGCGTCAG
CGGGTGTTGGCGGGTGTCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGATTGGCTAT
SEQ ID NO: 68: Receptor binding domain (RBD) use for P0055
RVQPTESIVRFPNITNLCPFGEVFNATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNF
SEQ ID NO: 69: T4 Fibrinitin trimerization domain
GYIPEAPRDGQAYVRKDGEWVLLSTFL
SEQ ID NO: 70: Linker sequence
GGGSSGGGSG
SEQ ID NO: 71: Linker sequence
GGGSS
SEQ ID NO: 72: Linker sequence
GGGSSGGGSSGGGSS
SEQ ID NO: 73: Linker sequence
GLGGL
SEQ ID NO: 74: Linker sequence
GLGGLAAA

1. A fusion polypeptide comprising
i) at least one antigenic unit;
ii) at least one antigen presenting cell (APC) targeting unit, which is targeting dendritic cells;
iii) optionally a multimerization unit, which unit provides for the multimerization of said fusion polypeptide to comprise two or more antigenic units and two or more antigen presenting cell (APC) targeting units,
wherein the APC targeting unit targets CCR7 and/or wherein the APC targeting unit targets mature dendritic cells (mDCs).

2. The fusion polypeptide according to claim 1, wherein the antigenic unit consist of or comprises a sequence of amino acids of at least one epitope being a neo-epitope of neoplastic cells from a patient.

3. The fusion polypeptide according to claim 1, wherein the antigenic unit does not comprise a neo-epitope of neoplastic cells from a patient.

4. The fusion polypeptide according to claim 1, wherein the antigenic unit is a bacterial or viral antigen.

5. The fusion polypeptide according to claim 1, which APC targeting unit is targeting mature dendritic cells (mDCs).

6. The fusion polypeptide according to claim 1, which APC targeting unit is selected from CCL19 and CCL21, including the human forms thereof.

7. The fusion polypeptide according to claim 1, which APC targeting unit is targeting the receptor CCR7.

8. (canceled)

9. (canceled)

10. (canceled)

11. The fusion polypeptide according to claim 1, wherein the APC targeting unit consists of or comprises an antibody binding region with specificity for target surface molecules on antigen presenting cells.

12. (canceled)

13. (canceled)

14. (canceled)

15. The fusion polypeptide according to claim 1, wherein the antigenic unit is connected to the targeting unit through a linker.

16. The fusion polypeptide according to claim 15, wherein the linker is or comprises a hinge region.

17. The fusion polypeptide according to claim 15, wherein the linker comprises a carboxyterminal C domain (CH3 domain).

18. The fusion polypeptide according to claim 17, wherein a hinge region and CH3 domains are connected by a sequence of amino acids comprising GGGSS (SEQ ID NO: 66), or wherein the hinge region and CH3 domains are linked via an amino acid sequence GGGSSGGGSG (SEQ ID NO: 70).

19. The fusion polypeptide according to claim 15, wherein the linker comprises a dimerization motif or any other multimerization domain, which participate in the multimerization through hydrophobic interaction.

20. The fusion polypeptide according to claim 15, wherein the linker comprises a hinge region comprising h1+h4 or h4 derived from IgG.

21. The fusion polypeptide according to claim 1, wherein the at least one antigenic unit consist of or comprises at least or about 5 epitopes.

22. The fusion polypeptide according to claim 1, wherein the at least one antigenic unit comprises at least one epitope, which exhibits an MHC binding stability, which is above average.

23. The fusion polypeptide according to claim 4, wherein the bacterial or viral antigen, which exhibits an MHC binding stability, which is above average, among the antigens of the respective bacteria or virus.

24. The fusion polypeptide according to claim 23, wherein the antigenic unit comprises an amino acid sequence derived from a β-corona virus.

25. The fusion polypeptide according to claim 24, wherein the amino acid sequence comprises or constitutes the Ribosome binding protein (RBD) from the SARS-CoV-2 spike protein.

26. The fusion polypeptide according to claim 25, wherein the amino acid sequence consists of or comprises residues 319-541 of NCBI reference sequence no: YP_009724390 or an amino acid sequence having at least 80% sequence identity therewith.

27. The fusion polypeptide according to claim 1, which multimerization, enables the formation of dimers, trimers, tetramers, pentamers, or multimers of higher order.

28. An expression vector, which comprises a sequence of nucleotides encoding a fusion polypeptide as defined in claim 1.

29. The expression vector according to claim 28, which sequence of nucleotides is a cDNA sequence or an RNA sequence.

30. (canceled)

31. The expression vector according to claim 28, which sequence further comprises or encodes at least one immune stimulating sequence (ISS).

32. The expression vector according to claim 31, wherein the ISS is an oligodeoxyribonucleotide (ODN) comprising at least one CpG motif, and wherein the ODN optionally includes phosphorothioate groups.

33. The expression vector according to claim 31, wherein the ISS is or comprises an oligoribonucleotide.

34. A system of at least two expression constructs comprising i) a first expression construct comprising a sequence of nucleotides encoding at least one antigenic unit, and ii) a second expression construct comprising a sequence of nucleotides encoding at least one antigen presenting cell (APC) targeting unit, which is targeting dendritic cells wherein the APC targeting unit targets CCR7 and/or wherein the APC targeting unit targets mature dendritic cells (mDCs).

35. The system of at least two expression constructs according to claim 34, wherein the first expression construct comprises a sequence of nucleotides encoding at least one antigenic unit, which
1) consists of or comprises a sequence of amino acids of at least one epitope being a neo-epitope of neoplastic cells from a patient, or
2) does not comprise a neo-epitope of neoplastic cells from a patient, or
3) is a bacterial or viral antigen, or
4) consists of or comprises at least or about 5 epitopes,
4) comprises at least one epitope, which exhibits an MHC binding stability, which is above average, or
6) is a bacterial or viral antigen, or comprises an amino acid sequence derived from a β-corona virus, or
7) comprises or constitutes the Ribosome binding protein (RBD) from the SARS-CoV-2 spike protein, or
8) consists of or comprises residues 319-541 of NCBI reference sequence no: YP_009724390 or an amino acid sequence having at least 80% sequence identity therewith.

36. The system of at least two expression constructs according to claim 34, wherein the second expression construct comprises a sequence of nucleotides encoding at least one antigen presenting cell (APC) targeting unit, which is targeting mature dendritic cells.

37. The system of at least two expression constructs according to claim 34, wherein the first expression construct comprising a sequence of nucleotides encoding at least one antigenic unit further comprises a sequence of nucleotides encoding a multimerization unit, which provides for the multimerization of said at least one antigenic unit.

38. The system of at least two expression constructs according to claim 34, wherein the at least two expression constructs are expressed on the same expression vector, optionally under the control of two different promotors.

39. The system of at least two expression constructs according to claim 34, wherein the at least two expression constructs are expressed by at least two different vectors.

40. A method for the treatment of a disease characterized by exhibiting a specific disease antigenic unit in the form of an epitope that is not exhibited by normal cells in the patient, or which epitope is foreign to the patient, the method comprising administering an immunogenically effective amount of a composition comprising
a) a fusion polypeptide as defined in claim 1, or
b) at least one expression vector as defined claim 28, or the system of at least two expression constructs according to claim 34, whereby somatic cells in the patient are brought to express the sequence of nucleotides contained within the expression vector; the method optionally further comprising administering a pharmaceutically acceptable carrier, diluent, or excipient.

41. The method according to claim 40, wherein the disease is a malignant neoplasm, wherein the neoplasm exhibits epitopes (neo-epitopes) that are not exhibited by non-neoplastic cells in the patient.

42. The method according to claim 40, wherein the disease is a bacterial or viral infection, which bacteria or virus is characterized by exhibiting a specific antigenic unit of said bacteria or virus.

43. The method according to claim 40, wherein the patient is a human being.

44. (canceled)

45. (canceled)

46. (canceled)

47. (canceled)

48. (canceled)

49. (canceled)

50. (canceled)

51. The method according to claim 40, wherein the immunogenically effective amount of a composition further comprises an effective amount of an amphiphilic block co-polymer comprising blocks of poly(ethylene oxide) and polypropylene oxide).

52. (canceled)


SEQUENCE LISTING (74 sequences)

SEQ ID NO: 1 - protein, 6 aa, Artificial sequence
  Artificial linker
GSGGGA

SEQ ID NO: 2 - protein, 12 aa, Artificial sequence
  Artificial linker
GSGGGAGSGGGA

SEQ ID NO: 3 - protein, 18 aa, Artificial sequence
  Artificial linker
GSGGGAGSGGGAGSGGGA

SEQ ID NO: 4 - protein, 24 aa, Artificial sequence
  Artificial linker
GSGGGAGSGGGAGSGGGAGSGGGA

SEQ ID NO: 5 - protein, 10 aa, Artificial sequence
  Artificial linker
GENLYFQSGG

SEQ ID NO: 6 - protein, 12 aa, Artificial sequence
  Artificial linker
KPEPKPAPAPKP

SEQ ID NO: 7 - protein, 12 aa, Artificial sequence
  Artificial linker
AEAAAKEAAAKA

SEQ ID NO: 8 - protein, 8 aa, Artificial sequence
  Artificial linker
SACYCELS

SEQ ID NO: 9 - protein, 10 aa, Artificial sequence
  Artificial linker
SGGGSSGGGS

SEQ ID NO: 10 - protein, 10 aa, Artificial sequence
  Artificial linker
GGGGSGGGGS

SEQ ID NO: 11 - protein, 10 aa, Artificial sequence
  Artificial linker
SSGGGSSGGG

SEQ ID NO: 12 - protein, 10 aa, Artificial sequence
  Artificial linker
GGSGGGGSGG

SEQ ID NO: 13 - protein, 10 aa, Artificial sequence
  Artificial linker
GSGSGSGSGS

SEQ ID NO: 14 - DNA, 20 bp, Artificial sequence
  ODN sequence
ggggtcaacgttgagggggg

SEQ ID NO: 15 - DNA, 20 bp, Artificial sequence
  ODN sequence
gggggacgatcgtcgggggg

SEQ ID NO: 16 - DNA, 21 bp, Artificial sequence
  ODN sequence
ggggacgacgtcgtggggggg

SEQ ID NO: 17 - DNA, 20 bp, Artificial sequence
  ODN sequence
tccatgacgttcctgatgct

SEQ ID NO: 18 - DNA, 20 bp, Artificial sequence
  ODN sequence
tccatgacgttcctgacgtt

SEQ ID NO: 19 - DNA, 24 bp, Artificial sequence
  ODN sequence
tcgtcgttttgtcgttttgtcgtt

SEQ ID NO: 20 - DNA, 22 bp, Artificial sequence
  ODN sequence
tcgtcgttgtcgttttgtcgtt

SEQ ID NO: 21 - DNA, 23 bp, Artificial sequence
  ODN sequence
tcgacgttcgtcgttcgtcgttc

SEQ ID NO: 22 - DNA, 26 bp, Artificial sequence
  ODN sequence
tcgcgacgttcgcccgacgttcggta

SEQ ID NO: 23 - DNA, 22 bp, Artificial sequence
  ODN sequence
tcgtcgttttcggcgcgcgccg

SEQ ID NO: 24 - DNA, 25 bp, Artificial sequence
  ODN sequence
tcgtcgtcgttcgaacgacgttgat

SEQ ID NO: 25 - DNA, 29 bp, Artificial sequence
  ODN sequence
tcgcgaacgttcgccgcgttcgaacgcgg

SEQ ID NO: 26 - DNA, 75 bp, Artificial sequence
  pTVG4 fragment sequence
agatctaacgacaaaacgacaaaacgacaaggcgccagatctggcgtttcgttttgtcgttttgtcgttagatct

SEQ ID NO: 27 - DNA, 20 bp, Artificial sequence
  CpG sequence
ggtgcatcgatgcagggggg

SEQ ID NO: 28 - DNA, 83 bp, Artificial sequence
  CpG sequence
ggtgcatcgatgcaggggggtatatatatattgaggacaggttaagctccccccagcttaacctgtccttcaatatatatata

SEQ ID NO: 29 - DNA, 4479 bp, Artificial sequence
  Plasmid sequence
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 30 - DNA, 10 bp, Artificial sequence
  Enhancer sequence
cgccgccacc

SEQ ID NO: 31 - DNA, 36 bp, Artificial sequence
  Dimer hinge encoding sequence
gagctcaaaaccccacttggtgacacaactcacaca

SEQ ID NO: 32 - DNA, 45 bp, Artificial sequence
  Dimer hinge encoding sequence
gagcccaaatcttgtgacacacctcccccgtgcccaaggtgccca

SEQ ID NO: 33 - DNA, 30 bp, Artificial sequence
  Dimer linker encoding sequence
ggcggtggaagcagcggaggtggaagtgga

SEQ ID NO: 34 - DNA, 321 bp, Artificial sequence
  Dimer CH3 encoding sequence
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 35 - DNA, 15 bp, Artificial sequence
  Dimer linker encoding sequence
ggcctcggtggcctg

SEQ ID NO: 36 - DNA, 276 bp, Artificial sequence
  Target unit encoding sequence
atgaaggtctccaccactgcccttgctgttcttctctgtaccatgacactctgcaaccaagtcttctcagcgccatatggagctgacaccccgactgcctgctgcttctcctacagccggaagattccacgccaattcatcgttgactattttgaaaccagcagcctttgctcccagccaggtgtcattttcctgactaagagaaaccggcagatctgcgctgactccaaagagacctgggtccaagaatacatcactgacctggaactgaatgcc

SEQ ID NO: 37 - DNA, 270 bp, Artificial sequence
  Target unit encoding sequence
atgaagctctgcgtgtctgccctctctctcctcttgctcgtggctgccttctgtgctccagggttctcagcaccaatgggctctgaccctcccacttcctgctgtttctcttacacctcccggcagcttcacagaagctttgtgatggattactatgagaccagcagtctttgctccaagccagctgtggtattcctgaccaaaagaggcagacagatctgtgctaaccccagtgagccctgggtcactgagtacatgagtgacttggag

SEQ ID NO: 38 - DNA, 273 bp, Artificial sequence
  Target unit encoding sequence
atgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagc

SEQ ID NO: 39 - DNA, 324 bp, Artificial sequence
  Target unit encoding sequence
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 40 - DNA, 342 bp, Artificial sequence
  Target unit encoding sequence
atgagacttctcctcctgactttcctgggagtctgctgcctcaccccatgggttgtggaaggtgtggggactgaagtcctagaagagagtagctgtgtgaacttacaaacccagcggctgccagttcaaaaaatcaagacctatatcatctgggagggggccatgagagctgtaatttttgtcaccaaacgaggactaaaaatttgtgctgatccagaagccaaatgggtgaaagcagcgatcaagactgtggatggcagggccagtaccagaaagaacatggctgaaactgttcccacaggagcccagaggtccaccagcacagcgataaccctgactggg

SEQ ID NO: 41 - DNA, 291 bp, Artificial sequence
  Target unit encoding sequence
atggcctgcggtggcaagcgtctgctcttccttgctttggcatgggtactgctggctcacctctgcagccaggcagaagcagcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatg

SEQ ID NO: 42 - DNA, 399 bp, Artificial sequence
  Target unit encoding sequence
atggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagagga

SEQ ID NO: 43 - DNA, 423 bp, Artificial sequence
  Target unit encoding sequence
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 44 - DNA, 55 bp, Artificial sequence
  Secretion signal encoding sequence
atgaaatgggtgacctttctgctgctgctgtttgtgagcggcagcgcgtttagcg

SEQ ID NO: 45 - DNA, 785 bp, Artificial sequence
  Target unit encoding sequence
cggcccagccggccatggcggactacaaacaggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctaggtggtggtggtggttctggtggtggtggatccggcggcggcggctctggcggcggcggctctgaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctcggcctcgggggcc

SEQ ID NO: 46 - DNA, 831 bp, Artificial sequence
  Target unit encoding sequence
gcggcccagccggccatggcggactacaaacaggctgttgtgactcaggaatcagcactcaccacatcacctggtgaaacagtcacactcacttgtcgctcaagtaaaagcagccagagcgtgctgtatgatgaaaacaaaaaaaactatctggccaactgggtccaagaaaaaccagatcatttattcactggtctaataggttgggcgagcaccggcgaaagcaaccgagctccaggtgttcctgccagattctcaggctccctgattggagacaaggctgccctcaccatcacaggggcacagactgaggatgaggcaatctatttctgttattatgattttccgccgttcggcagtggaaccaaggtcactgtcctaggtggtggtggtggttctggtggtggtggatccggcggcggcggctctggcggcggcggctctgaggtccagctgcaacagtctggacctgtgctggtgaagcctggggcttcagtgaagatgtcctgtaaggcttctaacgcggcgatttatatgcactggatgaaacagagccatggaaagagtcttgagtggattggaattcgcattcgcacccgcccgagcaaatatgcgaccgattatgcggatagcgtgcgcggcagctacaaccagaaattcaagggcaaggccacattgactgttgacaagtcctccagcacagcctacatggagctcaacagcctgacatctgaggactctgcagtctattactgtcgcgcgaccgaagatgtgccgttttattactggggccaaggcaccactctcacagtctcctcggcctcgggggcc

SEQ ID NO: 47 - DNA, 36 bp, Artificial sequence
  Target unit encoding sequence
tggcccaggttccacagcagcgtgttccacacccac

SEQ ID NO: 48 - DNA, 489 bp, Artificial sequence
  Target unit encoding sequence
atgacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcag

SEQ ID NO: 49 - DNA, 105 bp, Artificial sequence
  Dimer encoding sequence
ggagaactggaggaattacttaaacatctcaaggagttgctcaaaggccctaggaagggagaactggaggaactcctcaaacatctcaaggagttactaaaggga

SEQ ID NO: 50 - DNA, 30 bp, Artificial sequence
  Dimer encoding sequence
ggcagcggcagcggcagcggcagcggcagc

SEQ ID NO: 51 - DNA, 333 bp, Artificial sequence
  Dimer encoding sequence
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 52 - DNA, 180 bp, Artificial sequence
  Trimer encoding sequence
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 53 - DNA, 117 bp, Artificial sequence
  Tetramer encoding sequence
aagcctctggacggagagtatttcactctccagatccggggccccgaaaggttcgaaatgttccgggagcttaacgaggccttggagctgaaagacgcacaggccggaaaggaaccg

SEQ ID NO: 54 - protein, 27 aa, Artificial sequence
  Synthetic sequence
QIETQQRKFKASRASILSEMKMLKEKR

SEQ ID NO: 55 - protein, 27 aa, Artificial sequence
  Synthetic sequence
VILPQAPSGPSYATYLQPAQAQMLTPP

SEQ ID NO: 56 - protein, 27 aa, Artificial sequence
  Synthetic sequence
DTLSAMSNPRAMQVLLQIQQGLQTLAT

SEQ ID NO: 57 - protein, 27 aa, Artificial sequence
  Synthetic sequence
DGQLELLAQGALDNALSSMGALHALPR

SEQ ID NO: 58 - protein, 27 aa, Artificial sequence
  Synthetic sequence
RLHVVKLLASALSTNAAALTQELLVLD

SEQ ID NO: 59 - protein, 12 aa, Artificial sequence
  Dimer sequence
ELKTPLGDTTHT

SEQ ID NO: 60 - protein, 15 aa, Artificial sequence
  Dimer sequence
EPKSCDTPPPCPRCP

SEQ ID NO: 61 - protein, 27 aa, Artificial sequence
  Neoepitope
QIETQQRKFKASRASILSEMKMLKEKR

SEQ ID NO: 62 - protein, 27 aa, Artificial sequence
  Neoepitope
VILPQAPSGPSYATYLQPAQAQMLTPP

SEQ ID NO: 63 - protein, 27 aa, Artificial sequence
  Neoepitope
DTLSAMSNPRAMQVLLQIQQGLQTLAT

SEQ ID NO: 64 - protein, 27 aa, Artificial sequence
  Neoepitope
DGQLELLAQGALDNALSSMGALHALPR

SEQ ID NO: 65 - protein, 27 aa, Artificial sequence
  Neoepitope
RLHVVKLLASALSTNAAALTQELLVLD

SEQ ID NO: 66 - protein, 5 aa, Artificial sequence
  Linker
GGGSS

SEQ ID NO: 67 - DNA, 5525 bp, Artificial sequence
  Plasmid construct
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgccgccaccatggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtctgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctggcggccgcgagggtgcagccgactgagagtatcgttcgtttccctaacattacgaacctatgtccgtttggagaggtatttaatgctaccagattcgcatcagtctatgcttggaacaggaaacggatatcaaactgcgtggccgactatagcgttctctacaacagtgcctcttttagcactttcaagtgctacggagtctccccaaccaagctaaacgacctgtgcttcacaaacgtctacgctgactcctttgtcatcagaggcgatgaggtgcggcaaatcgcacctggacaaactggaaaaatcgccgactataactataagctgcccgatgactttactggatgcgtgatcgcttggaattcaaacaatctcgacagtaaggtaggaggcaattacaactacctttatcggctcttccgaaaatcaaacctgaaaccctttgagcgtgatatctcgactgaaatttaccaagctggcagcactccttgcaacggagtggagggcttcaattgttactttcctctgcagagctacggattccaacctacaaacggggtaggctaccaaccctaccgagtggtggtgctgagctttgagctgctccatgctccagcaactgtatgcggccctaagaagtcaactaacctggttaagaataaatgcgtcaacttttagggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 68 - protein, 223 aa, Artificial sequence
  Receptor binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 69 - protein, 27 aa, Artificial sequence
  Trimerization domain
GYIPEAPRDGQAYVRKDGEWVLLSTFL

SEQ ID NO: 70 - protein, 10 aa, Artificial sequence
  Artificial linker
GGGSSGGGSG

SEQ ID NO: 71 - protein, 5 aa, Artificial sequence
  Artificial linker
GGGSS

SEQ ID NO: 72 - protein, 15 aa, Artificial sequence
  Artificial linker
GGGSSGGGSSGGGSS

SEQ ID NO: 73 - protein, 5 aa, Artificial sequence
  Artificial linker
GLGGL

SEQ ID NO: 74 - protein, 8 aa, Artificial sequence
  Artificial linker
GLGGLAAA